(19) United States
(12) Patent Application Publication
(10) Pub. No.: US 2017/0003264 A1
(43) Pub. Date: Jan. 5, 2017

(54) Volatile Hydrocarbon Separation and Analysis Apparatus and Methods

(71) Applicant: The University of Wyoming Research Corporation d/b/a Western Research Institute, Laramie, WY (US)

(72) Inventors: Jeramie J. Adams, Laramie, WY (US); John F. Schabron, Laramie, WY (US)

(21) Appl. No.: 15/113,735

(22) PCT Filed: Jan. 24, 2014
(86) PCT No.: PCT/US14/13021

§ 371 Date: Jul. 22, 2016

(51) Int. Cl. G01N 33/28

(52) U.S. Cl. CPC G01N 33/2823

(57) ABSTRACT

At least one embodiment of the inventive technology may be described as a method for analyzing a hydrocarbon that comprises volatiles, said method comprising the steps of: segregating said volatiles from said hydrocarbon without oxidizing said hydrocarbon; generating a hydrocarbon residue and segregated hydrocarbon volatiles; and analyzing at least one of said hydrocarbon residue and said segregated hydrocarbon volatiles. The advantageous avoidance of oxidation may be achieved by placing the hydrocarbon under a vacuum, which may also enable the avoidance of cracking of the hydrocarbon while still achieving segregation of volatiles as desired. One other of the several embodiments disclosed and claimed herein may focus more on vacuum transfer and vacuum distillation of hydrocarbon volatiles. These and other methods disclosed herein may be used to achieve improved hydrocarbon analysis results.

### TECHNICAL FIELD AND BACKGROUND OF THE INVENTION

Knowing the chemical composition of hydrocarbons (including but not limited to biofuels, petroleum oils, shale oils, coal-derived oils, synthetic oils, vegetable or nut oils, oils from environmental releases, pollutant oils, lubrication oils, recycled oils, and asphalt materials) is critical in applications such as improving the performance of bituminous roadways as well as improving refining and oil production efficiency. Certain embodiments of the inventive technology disclosed herein combine innovative features that provide a comprehensive separation of oils in a manner that has not yet been achieved. This closed system quantitative vacuum distillation separation technology, in particular embodiments, provides a volatiles and non-volatiles fractions with minimal to no loss of volatiles, with temperatures below pyrolysis temperature (<340° C.), and an inert atmosphere to prevent oxidation. The volatiles and non-volatiles fractions can be characterized by a variety of techniques/analyses to provide information about the whole oil (or more generally hydrocarbon) sample. The generated data provide valuable insight into compositional differences between different oils and asphalt binders, internal chemical changes which occur due to aging or processing, changes during general processing and upgrading, and variability within wells. The results can be used to establish compatibility and for predictive modeling within reservoirs and along the production chain, to enhance/improve process control, to enhance catalyst use/efficiency, to improve processing efficiency and yields, to enhance efficiency of upgrading, and to track effectiveness of mitigation strategies, inter alia.

Adsorption Chromatography Petroleum Separations: For saturates, aromatics, resins, and asphaltene (SARA) separation of oils—due to irreversible adsorption onto typical column chromatography sorbents—the first step is to precipitate the most polar and pericondensed material using an aliphatic hydrocarbon solvent such as propane, pentane, hexane, heptane, or isooctane. The insoluble precipitate material is defined as asphaltenes. The amount of asphaltenes can vary significantly between methods since the amount of asphaltenes depends on solvent volume, temperature, time, and washing methods (Mitchell and Speight 1975, Andersen and Tenby 1996, Kharrat et. al. 2007).

The bulk of an oil is generally the soluble portion from an asphaltene precipitation which is called the maltenes and is typically separated further into saturates, aromatics, and resins fractions by normal phase liquid chromatography using column, thin layer, or rod chromatography. Separating a material into its constituent parts is often necessary in defining its composition and how the material will perform for particular applications or behave during transport, refining, upgrading, etc. Separations of oils using normal phase chromatography have been around for several decades. One early version of such type of analysis was developed by Corbett (1969) who separated asphalts into saturate, naphthene aromatic, polar aromatic and asphaltene fractions. A similar procedure was described by Jewel et al. (1972), in which crude oil or asphalt was separated into SARA fractions. The weight percent of each of the fractions was determined by drying the precipitated asphaltenes, and for the SAR fractions by evaporating the chromatography solvents and weighing the residual materials. These separation methods are limited to use with heavy oil materials such as residua and asphalt which do not contain significant volatile components that would be lost during the solvent evaporation step (Wu et. al. 2012). In most procedures there is no attempt made to control the amount of volatiles lost during the evaporation step. Crudes which contain significant lighter material can be topped by distilling a portion of the lightest hydrocarbons. The results from lab to lab vary significantly yielding significantly different results depending on the method used, which in turn leads to erroneous conclusions when using SARA analysis as a diagnostic and predictive method for crudes (Kharrat et. al. 2007).

Regarding quantification of SARA fractions without separating the fractions, there are reported methods based upon infrared (IR) and near infrared (NIR) spectroscopy which uses multivariate analysis to determine SARA components within a crude oil which was empirically derived from standard high performance liquid chromatography (HPLC) SARA separations data (Aske et. al. 2001). However, in general most data are still collected by separating the SARA fractions.

Rod Chromatography: Approaches for SARA separation can be divided into two main groups. The first method that has been widely utilized uses a technique known as thin-layer rod chromatography (TLC), and when combined with flame ionization detection (FID) becomes semi-automated. This is known as the Iatrocsan method in which capillary thin layer chromatography is conducted with whole oils on silica or alumina rods as a stationary phase, followed by evaporating the elution solvent and then slowly passing the rods through the flame of a FID to provide information on the relative amounts of the fractional zones on the rod (Jiang et al. 2008, Masson et al. 2001). The Iatrocsan system typically elutes the fractions in a sequence of solvents consisting of a linear alkane, cyclohexane, toluene, and dichloromethane:methanol mixtures. However, the Iatrocsan method has severe drawbacks including variable FID response factors for the different fractions, relatively high amounts of polar compounds are retained near the spot location on the TLC rod, aromatics group together to act like resins during separation, and it must be conducted on material that does not have any volatile material which boils below 220-260° C. (Fan and Buckley 2002). The separation is not very repeatable and there is a chronic problem arising from the strongly adsorbed, asphaltenic material which does not migrate up the rod. Improvements to this method require extensive calibration to adjust the response factor which can be accomplished by calibrating to SARA values obtained by open column or HPLC SARA separations (Orea et. al. 2002). However as with all SARA separations, for lighter crudes containing boiling components bellow 220-260° C. they must first be distilled, or topped, and the volatile fraction must still be analyzed by other methods to quantify the distillate (condensed volatiles) composition.

Column Chromatography: The second type of method is usually performed only on the maltenes; therefore, asphaltenes must first be separated by typical methods such as those described in ASTM D 3279, ASTM D 4124, ASTM D 2007 or similar. Many other variations of the SAR separation have been developed using amino, cyano, or alumina columns including several automated or semi-automated methods utilizing HPLC. Radke et al. (1980) described a semi-automated, medium pressure liquid chromatography system to separate maltenes involving three analytical columns and three pre-columns in which the pre-columns had to be re-packed between each injection. They used refractive index (RI) and optical absorbance detractors. In another example, Grizzle and Sablotny (1986) developed a method that used two aminosilane columns with a RI detector. Neither of these detector types provide uniform response for a variety of chemical types found in petroleum oils. The RI detector provides a response relative to the refractive index of the solvent. Components of oil vary greatly in their refractive indexes (Fan and Buckely 2002). An optical absorbance spectrometer detects compounds only with chromophores that absorb light at particular wavelengths, and different types of compounds either do not absorb much light or absorb at very different wavelengths of light, or have largely different molar absorptivities. These detectors are not best suited for providing quantitative results from the separations of components from complex systems such as petroleum.

An invention has been described which involves a novel combination of two modes of separation/analysis for hydrocarbons such as, e.g., bitumen and oils, including but not limited to petroleum oils, asphalt, coal liquids and shale oils. The technique is an automated on-column precipitation and re-dissolution solubility separation in which asphaltenes and/or waxes are precipitated within an inert stationary phase such as ground polytetrafluoroethylene (PTFE) packed in a column. This may be referred to as the Asphaltene Determinator™ (AD) or Waxphaltene Determinator™ (WD) separation, and may be as described in U.S. Pat. No. 7,875,464 (and derivative patents thereof, perhaps supplemented by disclosure herein, Schabron and Rovani 2008, Schabron et. al. 2010, Schabron et al. 2013). In the second component, the material which is not precipitated may be passed onto one or more series of adsorption chromatographic column(s) such as, but not limited to, automated or manual normal-phase adsorption liquid chromatography for separation into fractions such as saturates, aromatics, and resins. Another separation also includes the step of separating the asphaltenes into solubility subfractions by the Saturates, Aromatics, Resins separation combined with AD method (SAR-AD™) (Boysen and Schabron 2013). These separation systems all rely on evaporation of solvent used in the separations. The automated separations also can use an evaporative light scattering detector (ELSD) to provide near uniform responses to all of the components in the complex petroleum matrix. The percent ELSD area response is then assumed to be representative of the approximate weight percent of the separated materials.

A limitation to the any of the traditional SARA methods and also the automated AD, SAR-AD, and WD methods is that for samples containing volatile components, such as less than about C25 hydrocarbons, these are lost when the solvent is evaporated to measure the weights of the components, or are evaporated when the components are passed through a detector where evaporation occurs such as an ELSD or charged aerosol detector (CAD) (or other evaporation causing (or evaporative) analysis). Note that volatiles may be defined as including at least substantially all of those hydrocarbon components that, if subjected to whichever evaporative analysis is used for residue analysis, would evaporate when subjected to that analysis (whether as part of a hydrocarbon or as segregated therefrom). Accordingly, the C25 or less designation for volatiles might not be accurate for certain hydrocarbons (it might only apply to the case of hydrocarbons that include linear alkanes). For example, aromatics (e.g., C20) may not evaporate in the ELSD (and as such would not be appropriately included in the term volatiles) but a highly branched alkane that is C29 may. The volatiles definition typically will depend in large part on the structure of particularly smaller chained components, the number of heteroatoms (N, S, and O), and the type of residue analysis. As such, a more appropriate definition, which accounts for many different situations, characterizes volatiles as including at least those components that would evaporate if subjected to the residue analysis. In these cases, volatile material is lost and not detected, and there is a gap called “volatiles loss” in the data for the method. These losses can be up to 60 weight percent or more, depending on the sample or type of hydrocarbon. Use of a refractive index (RI) detector does not result in the loss of volatiles, but because the different components of oil have different refractive indexes greater than or less than the solvent used in the separations, quantification of components cannot be performed accurately with a RI detector. Another limitation of a RI detector is that since different solvents have significantly different refractive indexes, the use of a RI detector does not allow for solvent switching or gradients during the separation. The ELSD and CAD detectors do not suffer from this limitation. However they both suffer from the limitation that volatile components in the sample are evaporated with the solvent and are not detected.

For lighter crude oil the volatiles component of the oil can be a significant portion of the oil. This volatiles fraction is often enriched in saturates and contains some aromatics, which, depending on their ratio and type (liner vs. cyclic for saturates, and monoaromatic vs. polyaromatic and substituted vs. unsubstituted aromatics) have a significant influence on the stability of the asphaltenes which affects their behavior in the reservoir, during transportation, refining, upgrading, and other properties of the oil or residue. When predicting stability of oil by SARA fractionation or other methods, including but not limited to flocculation titration, open column SARA, SAR-AD, AD, WD, RI Detection or any other analysis technique, if the volatiles are unaccounted for the stability of the oil matrix can be significantly overestimated or underestimated, which can have adverse effects for predicting asphaltene precipitation, sediment formation, fouling in reservoir formations, fouling or corrosion in pipelines and storage units, fouling or corrosion of heat exchangers, settling, blending, emulsions, heat induced fouling, efficiency of production or processing, processing, upgrading, distillation yields, hydroprocessing, catalytic hydrocracking, atmospheric or vacuum distillation, delayed of fluid coking, determination of fuel or product properties from analysis of feeds or fuels, determination of value for chemical feedstock preparation, environmental spill characterization, and environmental remediation. The method can also be used to analyze other substances which may contain volatile portions where the heavier residual material can be subjected to other analysis or analysis which use an ELSD detector such as coal liquefaction, coal tar processing, coal liquid processing, fracking fluid analysis, biofuels, food grade oils, lubrication oils, cosmetics (not limited to soaps, shampoos, detergents, fragrances, and makeup), foods, drinks, biological samples, pharmaceuticals, medical diagnosis and treatment.

Another application of the distillation method is that it can be used as a convenient cut off point for determining total acid number of the distillate (or condensed volatiles) and residue—without losing volatiles—to aid in making determinations about where in the refinery corrosion is most likely to occur. Low boiling acids cause corrosion in pipelines and high boiling acids cause corrosion in heated (above 300° C.) portions of the refinery (Transportation Research Board Special Report 331, 2013).

For volatile petroleum, the volatiles fraction is often neglected when reporting SARA quantification (Kharrat et. al. 2007). Even when analyzing heavy crudes by open column chromatography SARA fractionation errors up to 20% have been reported and attributed to volatiles lost during solvent evaporation (Wu et. al. 2012). For this reason a volatiles tracking of SARA (VSARA) components was developed by tracking the weight change of the whole oil and each fraction upon repeated dissolution in isooctane followed by drying with a nitrogen stream (Wu et. al. 2012). Although the method shows some repeatability it takes long times to acquire the data and the composition of the initial volatiles from the whole oil are not identified. Another method to track the contribution of the volatiles employs a distillation process to remove the lightest volatiles with boiling points below 200-300° C. at atmospheric or reduced pressure to generate crude which has been topped and then the distillate is analyzed by gas chromatography/mass spectrometry (GC/MS) (Vazquez and Mansoori 2000) or HPLC (Orea et. al. 2002). There is no standard method for topping crudes, and often the method of distillation is not reported leaving out details as to how volatile loss was minimized and if any steps were taken to prevent oxidation of the remaining residue. One study which compares topping methods suggests that spinning band distillation is the most accurate but details concerning how to control volatiles loss and oxidation of the residue are not considered (Kharrat et. al. 2007). Distillation of petroleum to obtain native SARA quantification must be conducted in a controlled manner to prevent oxidation (often not considered in petroleum distillations) and bond cleavage through thermal cracking. In order that the native bulk content of the fluid is not significantly perturbed by thermal cracking the temperature must stay below about 340° C. Indeed, the goal (and indeed a step) of at least one embodiment of the inventive technology disclosed herein is to remove the volatiles from the hydrocarbon (and segregate the volatiles from the hydrocarbon, thereby generating residue and segregated volatiles, which are segregated from the residue) without cracking the hydrocarbon. The volatiles may be characterized as including at least substantially all components of the hydrocarbon that would be evaporated if subjected to the evaporative analysis used to analyze the residue, and that are actually evaporated during use of the inventive method. As such, the amount and character of the volatiles may change, even with the same hydrocarbon, depending on what sort of analysis is used on the residue. It should be understood that it is only necessary to remove from the hydrocarbon substantially all of the volatiles that would be evaporated if the hydrocarbon (in pre-treatment condition (i.e., without volatiles removed therefrom)) were subjected to the chosen evaporative analysis (chosen, for example, because it yields the desired parameter); note that such chosen analysis is, in the inventive technology, typically used on the residue. Note also that the composition of the oil can be catastrophically altered if the distillation procedure is not conducted under an inert atmosphere due to oxidation which consumes mainly aromatics and resins components and converts them into other resins or asphaltenes. Distillations conducted under atmospheric conditions must be thoroughly purged with an inert gas and be maintained under an inert atmosphere. Unless freeze-pump-thaw is used to degas the oil, purging with an inert atmosphere or having a flowing inert atmosphere during distillation procedure sweeps away volatile components. Distillations within closed systems are potentially dangerous since pressure build up can pop connecting joints of glassware or tubing causing damage, loss of samples, or injury.

For applications utilizing the ELSD, atmospheric distillation at 300° C. does not provide a deep enough cut to prevent volatiles loss. According to Robbins, to have 10% error within one standard deviation, 80% of the material must have a boiling point above 343° C. (1998), which has been verified by others (Khan and Brett 2004).

The current invention describes a vacuum transfer of the lightest volatiles (lighter portion of the volatiles) of petroleum crude oil followed by vacuum distillation to remove enough of the remaining volatile materials (heavier portion of the volatiles) to make the resulting residue amenable to any type of solvent evaporation or ELSD or CAD detection (examples of evaporative analyses), for example, with very little to no volatiles loss (because the volatiles have been removed from the residue) . In certain embodiments, vacuum transfer is used to remove a first portion of the volatiles from a hydrocarbon and vacuum distillation is used to remove a second portion. The volatile components of the first portion may have boiling points that are less than or equal to a first portion boiling point maximum, and volatile components of the second portion may have a second portion boiling point maximum that is higher than the first portion boiling point maximum; in certain cases, the first portion is a lighter portion (e.g., it has hydrocarbon chains with fewer carbons) of the volatiles and the second portion is a heavier portion (with hydrocarbon chains of more carbons) of the volatiles (in other cases, e.g., the second portion could be more aromatic or contain more heteroatoms). To prevent volatiles loss into the vacuum pump, liquid nitrogen (or, for example, another cryogenic fluid or solid) may be used to cool the collection flask which also facilitates the vacuum transfer of nearly all the volatiles while bringing the system under vacuum at ambient or slightly elevated temperatures. More viscous oils need to be gently warmed until they are fluid enough to be easily stirred with a stir bar. This method allows for efficient vacuum transfer of the lightest volatile components (the lighter volatiles portion) and can theoretically trap hydrocarbons as low as C2 and efficiently hydrocarbons which are about C3-C4 in length or longer (ethane boiling point is −89° C. and a melting point of −183° C. and nitrogen has a boiling point of −196° C.). In addition to preventing volatiles loss, liquid nitrogen prevents the volatiles from building pressure which can be dangerous and can contaminate the resulting residue when cooling the system after the distillation process. Liquid nitrogen may be used during the entire volatiles removal procedure in one example of this invention. Liquid helium could be used also, as could dry ice, dry ice baths, other cryogenic liquids or slurries, or electric chillers. When the volatiles are removed from the distillation flask an oil bath is applied to bring the temperature of the distillation flask up to 250° C. (higher temperatures can be obtained using other commercially available silicone oils). When the temperature of the vapor reaches a maximum and decreases by about 40-50° C. the distillation can be stopped. At an absolute vacuum pressure of 10 microns (0.01 mmHg), and assuming a perfectly insulated system, the theoretical maximum atmospheric equivalent boiling point is 598° C. In practice, we have observed that at 10 microns vacuum and when the distillation vapor temperature reaches about 145° C. (420-435° C. corrected boiling point, which is sufficient to collect saturated hydrocarbons in the range of C25-C29), the material which is distilled off results in a residue has virtually no ELSD volatiles loss. Some oils contain a significant amount of volatiles in the 145° C. range and in these cases distillation should be continued until no more volatiles are collected. For some oils surveyed the distillation maximum can be upwards of 162° C., while tar sands bitumens can be lower since they lack significant amounts of distillates in this region.

The distillation of crudes is paramount to the petroleum industry since the products they deliver are defined by the boiling point ranges of crude oil distillation. In a refinery setting, petroleum volatiles are initially subdivided into atmospheric and vacuum distillates. There are many methods, labs, and instruments which can determine or calculate boiling point distributions. ASTM D86-12 is a standard method to distill petroleum products at atmospheric pressure which is not applicable to products containing appreciable quantities of residual material and suffers from significant volatiles loss for lighter distillates. ASTM D2892-13 is a standard method using a reflux column to determine crude oil distillation at ambient pressure followed by switching to vacuum to reach a corrected boiling point of about 400° C. This method cannot accommodate petroleum mixtures with light ends such as light naphthas or mixture with initial boiling points above 400° C. It does however use dry ice traps to collect some of the lighter volatiles that bypass the condensers. To get higher distillation fractions ASTM D5236-13 gives a method to distill heavy hydrocarbon mixtures using a pot still at 0.5 mmHg to a corrected temperature up to 560° C., but it cannot accommodate petroleum fractions with boiling points lower than 160° C. and employs a flask skin temperature of 400° C. which is well within the pyrolysis region of petroleum. Gas chromatography methods are increasingly becoming more popular since the elution temperature can be converted to corrected temperatures and exist as standard methods ASTM D2887, D3710, and D7169. Others have attempted to vacuum distill oil at 0.75 mmHg but have lost naphtha material into the vacuum (Suzuki et. al. 1982). A high temperature gas chromatography (HTGC) method exits as D6352. However, no standard procedures are known to isolate and track volatiles for SARA or related analyses for lighter crudes with considerable volatiles (Kharrat et. al. 2007).

### SUMMARY OF THE INVENTION

When separating volatile oils such as crude oils, coal derived oils, fracking oils, and biofuels the volatile components are lost in the evaporation of solvent, as described above. This invention describes approaches to overcome that limitation to provide a more complete and repeatable SARA type analysis of oils containing volatile “light” components such as crude oils and biofuels. An initial separation step involves vacuum transfer followed by distillation in a sealed vacuum system which does not result in the loss of volatiles from the oil through the vacuum pump. The resulting distilled residuum can then be subjected to any typical analysis, such as SARA separations, in which the final step involves solvent evaporation with no significant loss of any component or the residuum sample material due to evaporation. The volatiles material collected in the sealed distillation step is then characterized by any method or technique. Particular embodiments of the inventive technology, disclosed herein, include a novel means of segregating these volatile materials from the sample before the analysis using any method that involves evaporation of solvent where there is potential for volatiles loss. The term segregating volatiles (or portion thereof) as used herein may imply not only removal of the volatiles, but preventing the removed volatiles from somehow entraining themselves back into the hydrocarbon or residue (in certain embodiments, where necessary, such prevention may be achieved by trapping the volatiles using a cooling bath around a collection vessel (e.g., collection flask) as described elsewhere herein).

Aspects of the inventive technology may also involve a novel combination of separation of volatiles from the whole sample oil containing volatiles and analysis of the volatiles fraction by proton or carbon nuclear magnetic resonance (NMR) spectroscopy (but one type of volatiles analysis). The NMR spectroscopy can utilize samples dissolved in CD₂Cl₂, a small reusable sealed capillary of CD₂Cl₂ immersed in the sample, or an NMR tube with a solvent capillary insert built in filled with CD₂Cl₂, or other appropriate non-interacting and non-interfering solvent. The experiments can be carried out with a flame sealed NMR sample tube or NMR tube fitted with a PTFE screw cap or any other type of cap that does not lose volatiles. Any other deuterated solvents which do not have interfering resonances in the aromatic or aliphatic region (most crude have very little to no resonances in the olefin region) can be used. The NMR spectroscopy can be proton or carbon NMR. From the NMR spectra, values such as aromaticity can be calculated (Lee and Glavincevski 1999, Rudzinski et. al. 2000, Buenrostro-Gonzalez et. al. 2001). Other structural features can be calculated based on well known calculation protocols. Some of these NMR calculation protocols require elemental analysis of the oils (carbon, hydrogen, nitrogen, sulfur, oxygen) (Clutter et. al. 1972, Lee et. al. 1990, Fossen et. al. 2011), which also can be conducted on the oils. Of particular interest may be distortionless enhancement by polarization transfer (DEPT) carbon (differentiates between CH₃, CH₂, CH, and quaternary carbons) NMR experiments and two dimensional DEPT carbon experiments correlated to proton NMR data which can help quantify carbons attached to aromatics and heteroatoms, branchiness of alkyl groups, naphthenic carbons, and quaternary carbons (Fossen et. al. 2011).

Another aspect involves analyzing the volatiles fraction by other types of volatiles analysis (e.g., one-dimensional, two-dimensional, or multi-dimensional gas chromatography (GC), gas chromatography/mass spectrometry, high performance liquid chromatography (HPLC) (Suatoni et. al. 1975, Robbins 1998, Aske 2001), or HPLC using a dielectric constant detector (Hayes and Anderson 1986)).

Another aspect involves analyzing the volatiles fraction by ultraviolet (Khan and Brett 2004, U.S. Pat. No. 4,988,446), infrared (Ramaswamy et. al. 1988, van de Ven, et. al. 1995, Aske et. al. 2001, Buenrostro-Gonzalez et. al. 2001), attenuated total reflectance infrared, Raman, Fourier transfer Raman (Michaelian et. al. 2001, de Peinder 2009), or most especially near infrared (Lysaght et. al. 1993, Maggard and Welch 1994a, 1994b, Aske et. al. 2001, Balabin and Safieva 2007) or acousto-optic tunable filter near infrared spectroscopy (Westbrook and Hutzler 1996).

Another aspect involves analyzing the volatiles fraction by optical fluorescence or turbidimetric spectroscopy.

Another aspect involves analyzing the volatiles fraction by X-ray fluorescence spectroscopy.

Another aspect involves analyzing the volatiles fraction by refractive index.

Another aspect involves analyzing the volatiles fraction by supercritical fluid chromatography and supercritical fluid extraction (Lee et. al 1990, Rudzinski and Aminabhavi 2000).

Another aspect involves analyzing the volatiles fraction by electrical resistivity (Bombardelli 2010).

Another aspect is the analysis of the non-volatile residue by a residue analysis such as on-column precipitation and re-dissolution methods such as but not limited to AD, WD, SAR-AD. Other analyses of the non-volatile residue fraction can include but at not limited to proton or carbon nuclear magnetic resonance (NMR) spectroscopy, one-dimensional, two-dimensional, or multi-dimensional gas chromatography (GC), GC/ mass spectrometry high performance liquid chromatography (MSHPLC), by spectroscopic techniques such as fluorescence, turbidimetric, ultraviolet, infrared, attenuated total reflectance infrared, Raman, Fourier transfer Raman, near infrared, or acousto-optic tunable filter near infrared spectroscopy.

Other aspects involve analyzing the non-volatiles residue fraction by high resolution Fourier transform ion cyclotron resonance mass spectrometry (FTICR/MS), optical or X-ray microscopy, X-ray fluorescence spectroscopy, measuring refractive index, supercritical fluid chromatography and supercritical fluid extraction, electrical resistivity, differential scanning calorimetry (DSC), thermogravimetric analysis (TGA), or flocculation titrations.

One advantage of at least one embodiment of the inventive technology is increased accuracy in results by capturing and analyzing volatiles material that otherwise becomes lost by evaporation during any analysis relative to amounts of constituents of an input hydrocarbon.

One advantage of at least one embodiment of the inventive technology is distilling a petroleum product so that its residue is amenable to analysis by ELSD with minimal volatiles loss.

One advantage of at least one embodiment of the inventive technology is an increase in distillate yield of a hydrocarbon that is analyzed (or, more particularly, a sample thereof that is analyzed). Such increase may stem from an enhanced or increased accuracy of results.

One advantage of at least one embodiment of the inventive technology is a method for interpreting aromaticity data for the distillate (or condensed volatiles) compared to SARA analysis of the residue (i.e., the hydrocarbon that remains after the volatiles are removed from it), preferably by open column SARA, AD, SAR-AD, and WD.

One advantage of at least one embodiment of the inventive technology is a method to use the volatiles fraction combined with fractionation of the vacuum distillation residue by open column SARA, ultraviolet, HPLC, infrared, and near infrared methods for determining SARA fractions, AD, SAR-AD, and WD methods to determine stability of oils with very low asphaltene content which are not amenable to traditional Heithaus or other flocculation titration methods.

One advantage of at least one embodiment of the inventive technology is that it can be used to determine if olefins are present in the oil, distillate, or the residue, or determine the amount of olefins present.

One advantage of at least one embodiment of the inventive technology is that it can be used to determine the stability of pyrolytic processes such as thermal cracking, hydrocracking, catalytic cracking, coking, and thermal upgrading processes.

One advantage of at least one embodiment of the inventive technology is that it may be used to determine or predict properties of a processed hydrocarbon based on analysis of the feed hydrocarbon.

One advantage of at least one embodiment of the inventive technology is that it can be used to determine if the oil is a bitumen or heavy oil blended with a light diluent or other light oil.

One advantage of at least one embodiment of the inventive technology is that it can be used to monitor thermal cracking and upgrading processes, such as visbreaking, coking, hydrotreating, fluidized catalytic cracking processes such as LC Fining, H-Oil, etc.

One advantage of at least one embodiment of the inventive technology is that the data gathered from the distillation and used in conjunction with SARA or other analysis wherein the volatiles are taken into consideration is predictability or control of oil phenomena or process conditions involving emulsions (e.g., emulsion formation, emulsion breaking, or emulsion formulation), heat exchanger fouling, distillation tower fouling, catalyst fouling, catalyst efficiency, fouling, settling, asphaltene deposition, asphaltene precipitation, sediment formation, distillation efficiency, coke formation, corrosion, asphaltene adsorption, etc.

One advantage of the at least one embodiment of the inventive technology is that it can be used to determine if the acids, particularly naphthenic acids, are more concentrated in the volatiles fraction or the residue helping to determine at which point in production, transportation, and refining corrosion will take place.

One advantage of at least one embodiment of the inventive technology is an increase in speed of analysis. Indeed, using certain embodiments of the inventive technology disclosed herein, time from input of a hydrocarbon sample to be analyzed to elution, analysis, and/or generation of results may be less than that found in conventional methods.

One advantage of at least one embodiment of the inventive technology is a reduction in polluting emissions (given a certain distillate yield or a certain hydrocarbon input to be processed).

An advantage of at least one embodiment of the inventive technology may be to generate improved analysis results and use those improved analysis results to improve predictability or control of one or more phenomena including but not limited to: emulsion-related effects, emulsion formation, emulsion formulation, breaking of emulsions, fouling generally, heat exchanger fouling, distillation tower fouling, catalyst fouling, catalyst efficiency, oil subfraction measurement, settling, asphaltene deposition, asphaltene precipitation, distillation efficiency, coke formation, corrosion, sediment formation and asphaltene adsorption.

An advantage of at least one embodiment of the inventive technology may be to use generate improved analysis results and use such results to improve control of at least one process; such process may be any process indicated herein or in any of the prior art documents incorporated herein by reference.

An advantage of at least one embodiment of the inventive technology may be to determine the stability of a pyrolytic process to which a hydrocarbon has been subjected.

An advantage of at least one embodiment of the inventive technology may be to characterize a hydrocarbon.

An advantage of at least one embodiment of the inventive technology may be to monitor a thermal cracking and upgrading process to which a hydrocarbon has been subjected.

An advantage of at least one embodiment of the inventive technology may be to determine relative concentrations of acid in the segregated hydrocarbon volatiles and hydrocarbon residue.

An advantage of at least one embodiment of the inventive technology may be to control, design and/or monitor processing of a hydrocarbon through use of information generated through use of any of the apparatus of any claim.

Other advantages of the inventive technology, in embodiments, may be as disclosed elsewhere in this specification, including the figures. Indeed, any effect or result of any of the various embodiments disclosed herein may be an advantage afforded by an embodiment of the inventive technology relative to the prior art.

### BRIEF DESCRIPTION OF THE DRAWINGS

FIG. 1 shows a diagram of the distillation apparatuses used. More particularly, it shows a diagram of the vacuum jacketed Vigreux vacuum distillation apparatus used for the vacuum transfer (to remove a first or lighter volatiles portion or fraction) and vacuum distillation (to remove a second or heavier volatiles portion or fraction) of crudes.

FIG. 2 shows a diagram of the preferred distillation apparatus in use with a cooling bath to trap the volatiles. More particularly, it shows a diagram of the distillation apparatus in use with DPMP-400 oil used to heat the oil and liquid nitrogen under the collection flask to trap the volatiles.

FIG. 3 shows a diagram of a sealed NMR tube containing a portion of volatiles material and a small sealed capillary filled with deuterated methylene chloride. More particularly, it shows a diagram of 5 mm NMR tubes fitted with Teflon valves (Chemglass, CG-512/ New Era NE-CAV5-M-170) containing distillate and a sealed glass capillary filled with CD₂Cl₂.

FIG. 4 shows the analysis tree for volatile crude analysis (volatile crude oil separation) by SAR, SARA, AD or SAR-AD analysis (open column).

FIG. 5 shows the ¹H NMR analysis of the distillate (condensed volatiles) portion of 7 different oils. More particularly, it shows ¹H NMR spectra of the aromatic (top), olefin (middle), and saturates (bottom) for volatiles vacuum transfer and vacuum distillation of crudes JS 1-JS3, Desalt Out 1, Desalt Out 2, Minnelusa, and Dilbit.

FIG. 6 shows a black box drawing of at least one apparatus embodiment of the inventive technology.

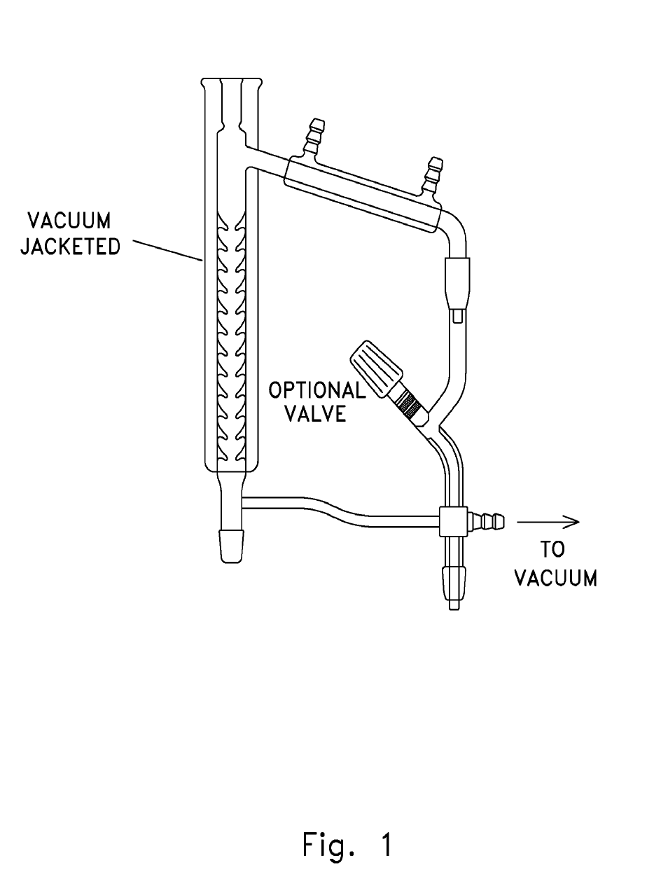

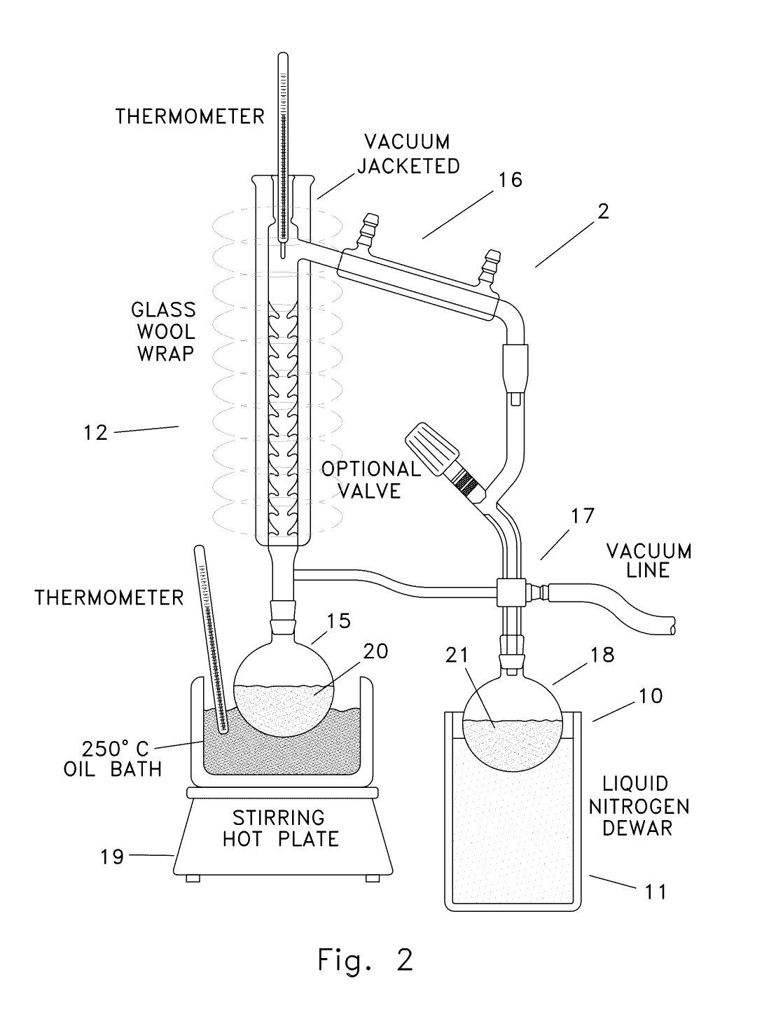

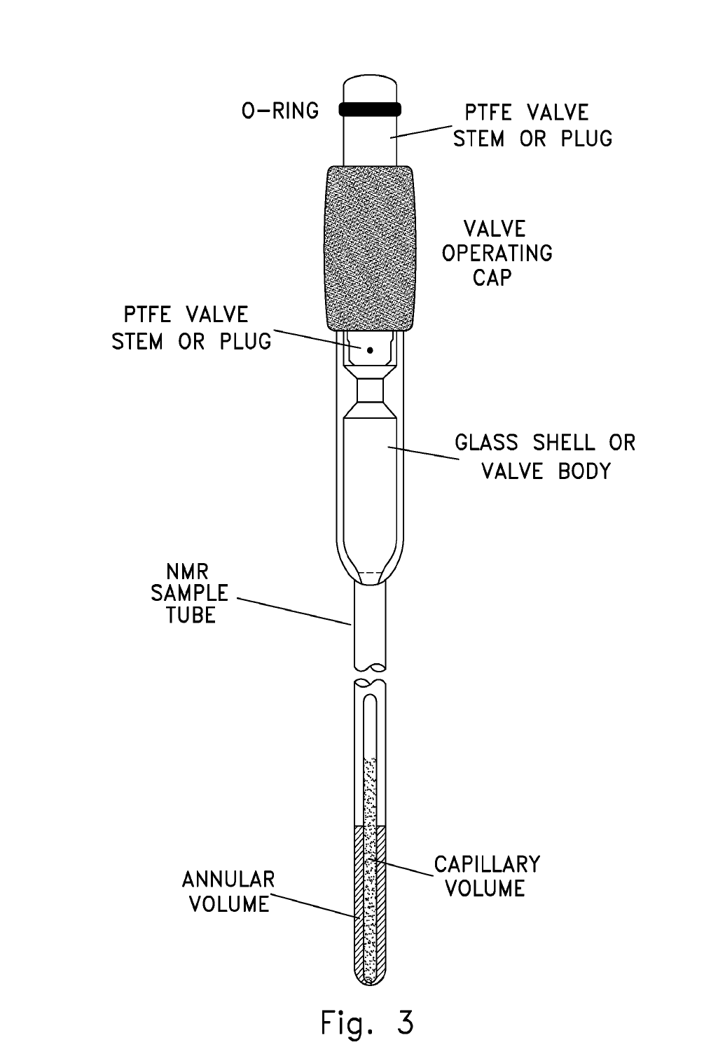

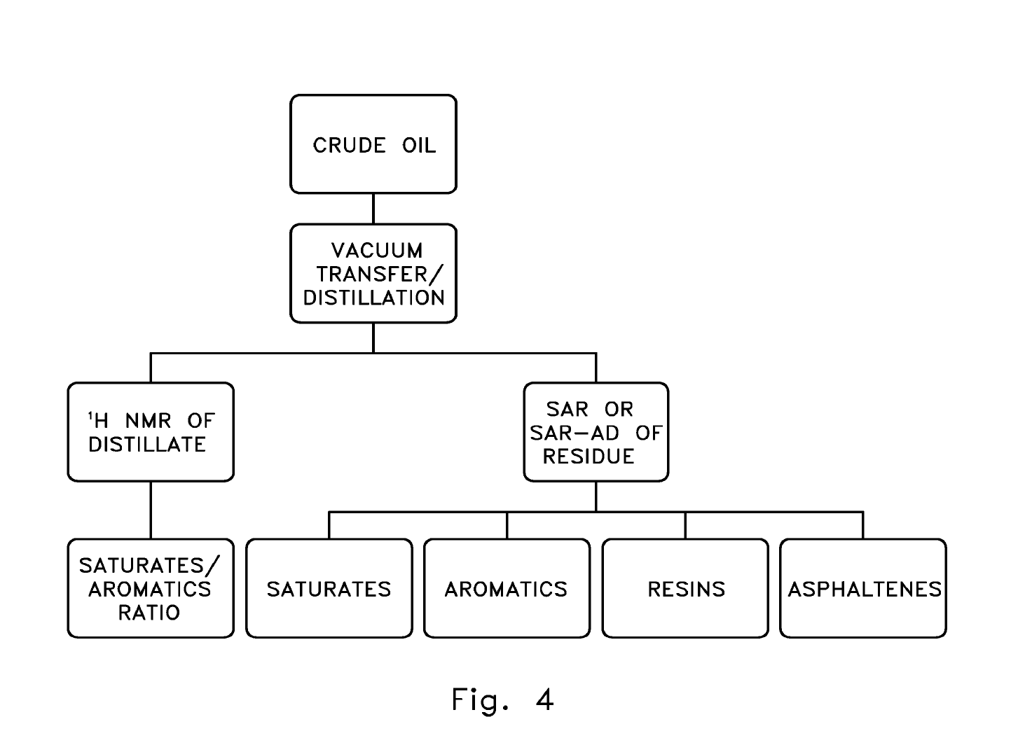

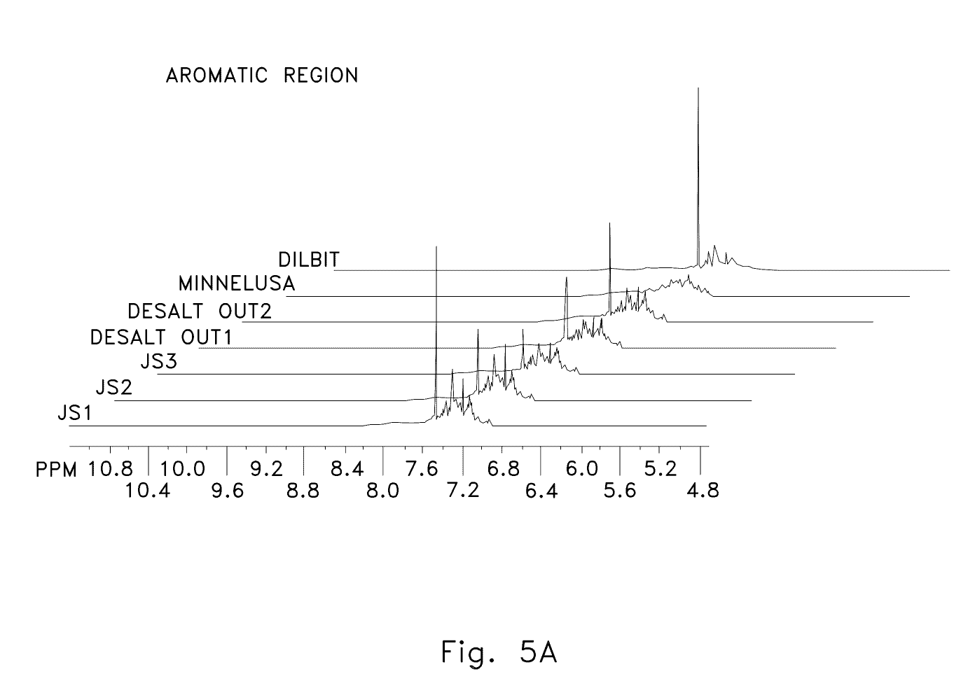

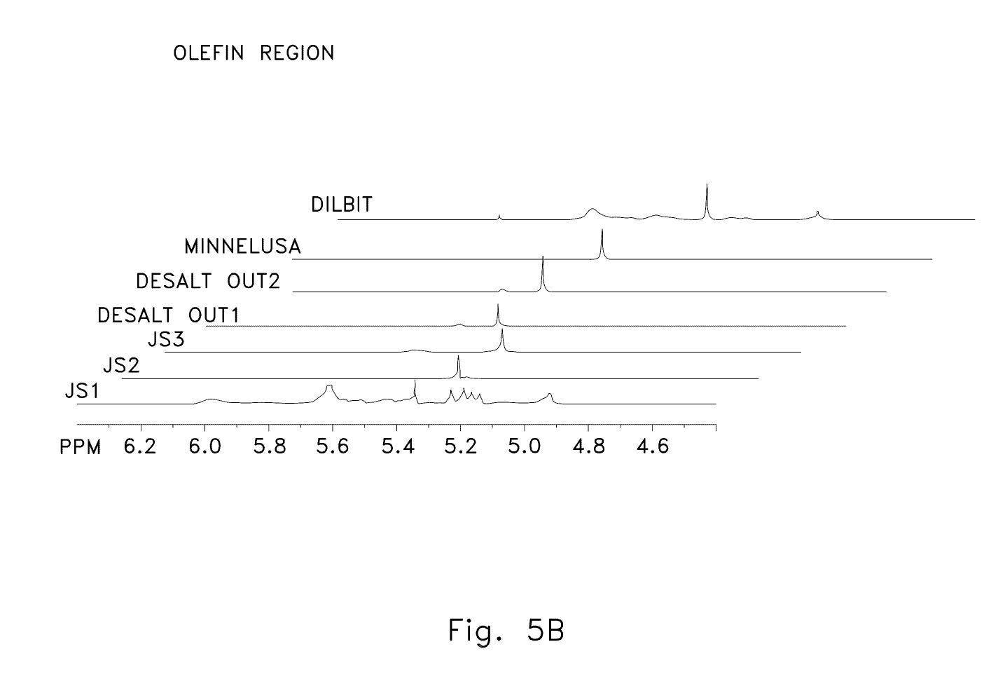

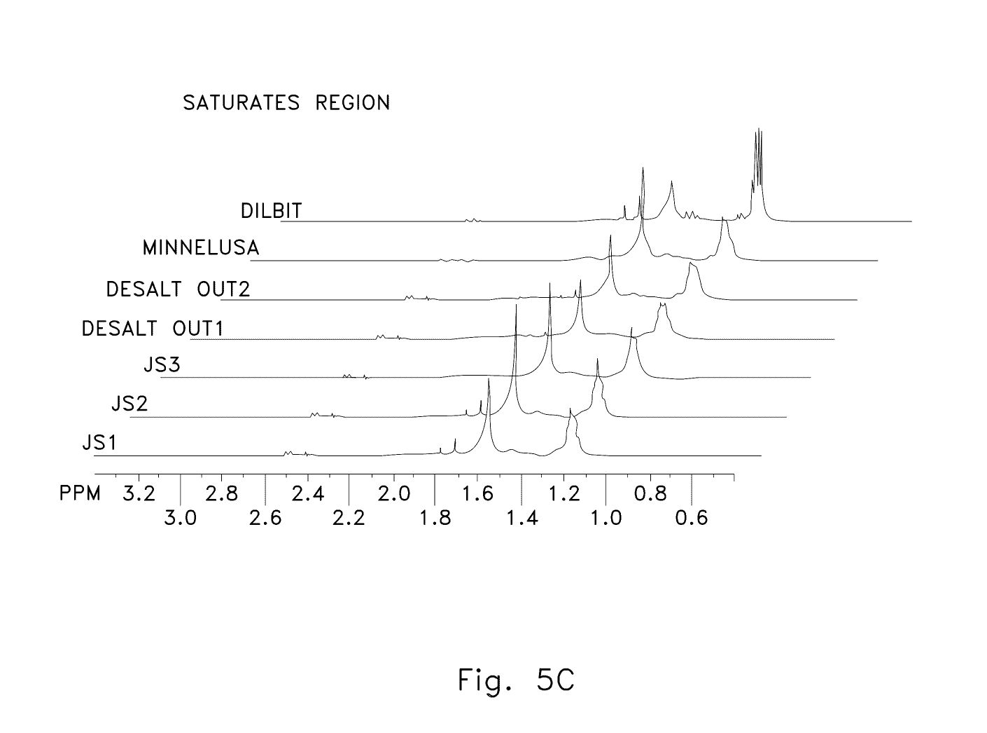

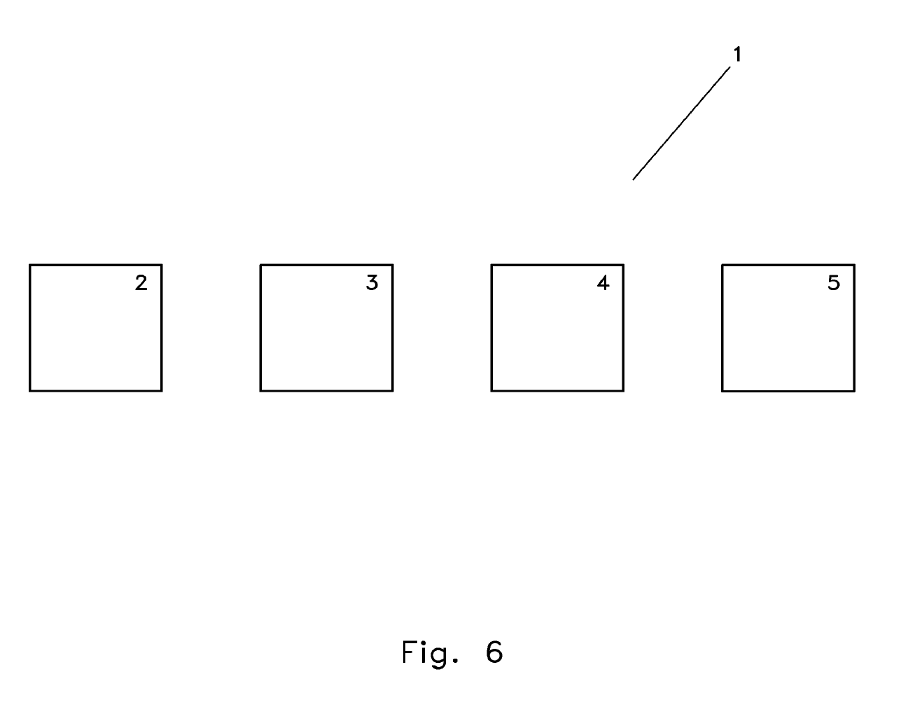

### DETAILED DESCRIPTION OF THE INVENTION

As mentioned earlier, the present invention includes a variety of aspects, which may be combined in different ways. The following descriptions are provided to list elements and describe some of the embodiments of the present invention. These elements are listed with initial embodiments, however it should be understood that they may be combined in any manner and in any number to create additional embodiments. The variously described examples and preferred embodiments should not be construed to limit the present invention to only the explicitly described systems, techniques, and applications. Further, this description should be understood to support and encompass descriptions and claims of all the various embodiments, systems, techniques, methods, devices, and applications with any number of the disclosed elements, with each element alone, and also with any and all various permutations and combinations of all elements in this or any subsequent application.

A substance, such as a hydrocarbon≧whether it be any of a variety of hydrocarbons, including but not limited to oils from fossil, biological or synthetic sources, or derived from biological/renewable oil sources (such as biomass), or recycled oil, or lubrication oil, or oil shale, or even coal (perhaps using a liquefaction or Fischer Tropsch process)≧may be established in a distillation vessel via any well known manners (injection, for example). A hydrocarbon may be, as but a few examples, oil, crude oil, a constituent of oil (e.g., maltenes), bitumen, binder (e.g., asphalt binder), light oil, medium crude, synthetic crude, heavy oil, dilbit, recycled oil, opportunity crudes such as heavy sour grades, oils and bitumen, extra heavy oil, high TAN crudes, and any of the other types of hydrocarbon mentioned in this specification, whether diluted in solvent solution or not. Applications also may include analysis of oils related to environmental pollution in soil, freshwater, or saltwater.

Vacuum distillation apparatuses can be purchased commercially such as #6563-10 from Ace Glass Incorporated (most similar to what was used), and #287320-0000 manufactured by Kontes, other related short path columns can be used as well such as #07-1440 from Specialty Glass Incorporated and others. After testing three different apparatuses it was determined that a distillation apparatus with a Vigreux vacuum jacketed distillation apparatus prevented carryover of any bumped or splattered undistilled oil as well as not having a place for a significant amount of distillate vapor to condense and collect. On the collection side of the distillation assembly a separation apparatus can be used to collect different boiling point fractions. It may be useful to have the distillation apparatus connected to a water source or circulating water or oil temperature bath so that the temperature of the distillation column can be increased to prevent waxes from solidifying in the condenser column. It was also found that insulating the vacuum jacketed Vigreux portion of the column by wrapping it with glass wool help to facilitate the distillation.

Prior to adding the flask containing the oil to be distilled, the distillation apparatus and collection flask can be evacuated and back flushed with nitrogen, or simply purged with nitrogen. Freeze-pump-thaw methods can also be employed when the flask containing the oil to be distilled is attached to the distillation apparatus. However in practice, during the initial pump down and vacuum transfer, when the distillation apparatus is assembled with the flaks containing the oil, we have found that when initially cooling small collection flasks (250 mL or less, larger flasks may also pose no problems) with liquid nitrogen before applying vacuum does not result in any appreciable condensing of ambient water or oxygen. Other liquids or means of cooling the collection flask can be used such as dry ice, dry ice slurries with solvents such as acetone, liquid nitrogen slurries, ice and salt baths, ice baths, circulating chillers, or any other method of cooling the collection vessel.

After the system is under full vacuum (5-40 microns, 0.005-0.04 mmHg) and the vacuum transfer of the lightest components (first volatiles portion) is completed, a stirred 200° C. diphenyl dimethyl silicon (DPDM-400 from Clearco) oil bath is gradually contacted with stirring the flask containing the oil which is under vacuum. Note: for heavier oils it is necessary to warm the flask up to 45-90° C. or more to facilitate adequate stirring and to prevent excessive bumping of the oil. As fractions of oil are removed and the vapor temperature of the distillate increases the level of the oil bath is gradual raised until the flask is covered about 3/4 of the way up. After most of the liquid has distilled at 200° C. the temperature of the oil bath is raised to 250° C. until the vapor temperature of the distillate reaches a maximum and decreases until about a 40° C. drop or more is observed and then the oil bath is removed. For our purposes a high vacuum line fitted with a high vacuum pump and oil diffusion pump was used however any source of vacuum capable of reaching less than about 0.05 mmHg absolute pressure will be suitable. Under a perfectly insulated system, to remove enough volatiles to render the distillation residue amenable for ELSD analysis, using an oil bath temperature of 300° C. and at a vapor temperature of 300° C., to get a corrected vapor temperature of at least 430° C. the minimum vacuum would need to be at least 30 mmHg. The heated source could be a heating mantle, other oil baths containing different high temperature oils, circulation baths, electrically resistive heated oil baths, aluminum block heaters, heating mantles or aluminum blocks with a sand bed, ovens, or other heating sources. The temperature of the heating medium should not exceed 340° C. to minimize cracking side reactions. However, in some cases where it is desirable to determine the stability of a pyrolyzed oil to determine liquid yields and coke formation, with or without various upgrading technologies, the bath temperature can be up to 500° C. with appropriate quartz glassware and glass coated stir bar or other appropriate stir bar or stirring mechanism since Teflon will decompose around 360° C. It is also necessary to use a suitable high performance high temperature grease such as Krytox™ fluorinated grease to seal the joints. Note that other metal types of distillation apparatus could be used.

The temperature of the distillate can be monitored using a calibrated thermometer of the correct immersion depth for the distillation apparatus. Thermocouples can also be used to measure the distillate vapor temperature. Thermometers or thermocouples can be inserted at different locations—distillation flask, distillation vapor exiting the distillation flask, distillation vapor prior to condenser column, vapor exiting the distillation column, or any other meaningful location—to monitor the progress of the distillation.

It is of note that the methods, and apparatus, described herein may be only separation methods or apparatus (where the goal is not to analyze a hydrocarbon relative to its constituent fractions, but instead to separate at least one constituent fraction thereof), or they may be only analysis methods (where the goal is not separation of at least one constituent fraction from a hydrocarbon, but rather analysis of a hydrocarbon, such as analysis of percentage composition of one or more of its constituent fractions), or they may be both (analysis and separation). Accordingly, while some embodiments may be analysis methods, some may be only separation methods (e.g., a method to separated/segregate volatiles from a hydrocarbon comprising the steps of vacuum transferring a lighter portion of the volatiles from the hydrocarbon and vacuum distilling a heavier (or second) portion of the volatiles from the hydrocarbon). Note that volatile components of the first portion may be described as having boiling points that are less than or equal to a first portion boiling point maximum, and volatile components of the second portion may be described as having a second portion boiling point maximum that is higher than the first portion boiling point maximum. Specifics as to actual values of these boiling points may be as indicated elsewhere herein.

Often, the purpose of any of the inventive methods disclosed herein is analysis of the input hydrocarbon (i.e., the pre-treatment hydrocarbon, or hydrocarbon in its condition before any of the volatiles are intentionally removed); typically, that analysis, in order to generate improved results, means a separation (segregation) of volatiles from the hydrocarbon to generate segregated volatiles and non-volatile residue. Results of the analysis thereof may be used to characterize in some manner (typically numerically) one or more of the various constituents of the input hydrocarbon (e.g., volatiles, saturates, aromatics, resins, naphthenes, asphaltenes, subfractions of polars, and solubility subfractions of asphaltenes, as but a few examples). Often, that characterization relates to the amount of the constituent(s) of interest in the hydrocarbon, whether on a percentage or other basis. Analysis of the non-volatile material (residue) following distillation (and perhaps a previous vacuum transfer), i.e., residue analysis, may include, but is not limited to well known evaporation of solvent followed by weighing, or separation using columns and weighing or using detectors, such as ELSD, optical absorbance or fluorescence, refractive index, CAD , and other spectrometers. Information gleaned from analysis (a solubility profile, as but one example) can additionally, or instead, aid in assessing compatibility of the oil or more generally hydrocarbon material associated with the input hydrocarbon (e.g., maltenes, or perhaps one containing asphaltenes), aid in conducting predictive modeling, aid in selecting feed (unprocessed hydrocarbon input) for process optimization, and aid in effecting process control and predicting properties such as emulsion or fouling or sediment formation propensity of feed or product oils, or in the product oils by analyzing the feed hydrocarbon. It is also of note that current methods, because of losses of volatiles, cannot achieve the accuracy afforded by the mentioned inventive technology. Regardless, refineries (a term that includes but certainly is not limited to laboratories that analyze hydrocarbons) using conventional technologies are processing hydrocarbons with limited information about them (e.g., about coking onset, blending, fouling, etc.) and their compositional makeup. As such, in order to avoid coke formation, or form only a small amount of coke during processing (or in order to avoid fouling of catalysts and/or heat exchangers, or only cause minimal fouling, or in order to avoid or minimize formation of emulsions in desalters, or to avoid or minimize corrosion, all during or as a result of processing), relatively conservative processing conditions are used. Indeed, the lack of information about the unprocessed (or partially processed) input hydrocarbon causes process operators to not produce as much end product(s) (e.g., gasoline, fuel oil, lubricating oils, diesel fuel, kerosene, jet fuel, tar, heavy fuel oil and asphalt) as could possibly be produced if they had more accurate, reliable information regarding compositional makeup, and could therefore “push”, or further adjust processing conditions (residence time, pressure, temperature, catalyst use, etc.), to produce more or higher quality product while still avoiding coke formation (or only forming an small amount of coke) or experiencing other undesired outcomes (e.g., any or too much fouling, unacceptable amounts of emulsion generation in desalters, catalyst fouling or deactivation). The more accurate the information, the more efficient the process is because, e.g., coke onset estimation becomes more accurate as a result. As such, particular embodiments of the inventive technology disclosed herein enable greater end product production, a supplemental end product, or an end product not produced using conventional technology for a given hydrocarbon processor input (refinery input). In this way, carbon dioxide and other undesired emissions (such as SOx, NOx, as but a few examples, all generally termed pollutants) can be reduced for a given production of a hydrocarbon end product (or a supplemental amount of oil can be produced for a certain amount of emissions, or for a given hydrocarbon processing expenditure, or for a given emissions allotment, allowance or expenditure). Such efficiency has obvious cost savings implications and, if a cap and trade scheme is legislated, will result in emissions credits associated with this “reduced emissions per produced end product” that, having a monetary value (estimated in 2011 to be from $20/ton to $140/ton, which may indeed change depending on the market conditions), can be traded on the market. Indeed, the owner of the inventive technology claims that market value, in addition to the supplemental oil per hydrocarbon input, or per emissions output afforded upon use of the inventive technology, inter alia.

As an example of calculations that suggest the magnitude of costs savings attributable to the inventive technology based on an estimate of 2.3 million barrels of heavy ends per day of thermal cracking and coker feed that can be produced from distillation operations in the U.S., an industry-wide 1% increase in distillate yield (end product) from safely cutting deeper into a heavy oil during distillation (perhaps a low end, conservative estimate) would result in about 23,000 bpd of supplemental end product, worth approximately $230,000/day, assuming a differential price between residua and distillate of $10/bbl. Further, there would be significant energy savings involved using aspects of the inventive technology, as coking operations use about 166,000-258,000 Btu per barrel of feed (USDOE 1998). For each 1% decrease in thermal cracking and coker feed (near 23,000 barrels per day in 2011, (USEIA 2011)), there would be a potential energy savings of about 3.8-5.9 billion Btu for residua that do not need to be heated for coking, since they will have been recovered in an optimized distillate stream. This also corresponds to a lowering of carbon dioxide from fuel that is not burned in coking operations. Residual fuel used as the heat source produces about 174 pounds of carbon dioxide per million Btu generated. Thus, in the U.S., the reduction in carbon dioxide emissions for each 1% industry-wide distillation efficiency improvement may be about 331-515 tons per day. Given the above-mentioned monetary per ton emissions estimate ($20-$140/ton), at 515 tons/day (188,000 tons/yr), which certainly could increase, market value for avoided CO₂ emissions (valued according to market value of traded emission credits) could be $3,760,000/yr up to $26,320,000/yr for each 1% gain in efficiency. So, a 5% efficiency gain would yield $18,800,000 to $131,600,000/yr in CO₂ emission value. Of course, actual savings/costs/value could be greater (including the 1% gain); these are merely estimates.

### Laboratory Results:

The following laboratory conditions and results, while presented using particular data, are not intended to limit the scope of the inventive technology.

Vacuum Transfer and Distillation. Prior to SAR-AD analysis of crudes JS1-JS3, Desalt Out 1, Desalt Out 2, Minnelusa, and Dilbit, the volatiles were vacuum transferred and vacuum distilled. A custom vacuum jacketed Vigreux vacuum distillation apparatus (nearly identical to #6563-10 from Ace Glass Incorporated) comprised of 14/24 flask connecting joints, a Teflon® valve, a properly jointed and immersion rated thermometer which read up to 300° C., and a pre-weighed empty 100 mL 14/24 round bottom receiver flask. All joints were lubricated with a small amount of high performance fluorinated Krytox™ grease and the distillation column was wrapped with glass wool. Water was not used to cool the condensing column because this can cause the waxes and other higher melting point material to solidify, circulating hot water is preferred. The distillation apparatus was attached to a high vacuum line capable of reaching an absolute vacuum of 5 microns (0.005 Torr). Approximately 35-60 g of crude oil was added to a pre weighted 14/24 100 mL round bottom flask fitted with a 1″ Teflon stir bar. The flask with oil was placed on the distillation apparatus (which may or may not be under nitrogen) and a Dewar containing liquid nitrogen was used to submerge the collection flask to about halfway. The collection flask was covered about halfway with liquid nitrogen throughout the entire distillation procedure to ensure that no volatiles were lost to the vacuum. To prevent over cooling of the collection flask joint a cloth was wrapped around where the top of the Dewar meet the collection flask and the liquid nitrogen level was checked and replenished often. After allowing the temperature of the collection flask to equilibrate for about 1-2 minutes vacuum was gradually applied until the oil bubbled gently and the vacuum was isolated. The lightest volatile material was allowed to vacuum transfer into the collection flask, as the bubbling ceased the vacuum was increased until gentle bubbling was observed and the vacuum was again isolated to allow the vacuum transfer of more volatiles. The vacuum transfer procedure was repeated until the system was under full vacuum at about 7 microns (0.007 Torr). After the system was under full vacuum a 200° C. diphenyl dimethyl silicon (DPDM) oil bath was slowly applied to the flask containing the oil with stifling. The DPDM silicon oil bath consisted of a 200 mL crystallization dish filled about halfway with DPDM-400 and fitted with a thermometer and a metal paperclip (as a flat stir bar); the crystallization dish was place on top of a stifling hot plate. The oil bath was gradually raised to touch the distillation flask. The distillation flask was allowed to equilibrate and some material was collected by distillation. After the collection of the distillate or condensed volatiles began to decrease, the oil bath height was gradually increased until the crude oil began to reflux and more material began distilling. The system was allowed to come to equilibrium and the oil bath was again raised incrementally as needed, it is necessary to raise the bath incrementally to avoid the crude from refluxing too vigorously, until the distillation flask was covered about 3/4 the way up with the 200° C. DPDM-400 silicon oil. The vacuum was periodically checked during the entire distillation process to ensure that there were no leaks. For some oils as the vapor temperature reached over 120° C. some material began to solidify in the condenser portion of the distillation apparatus and/or near the entrance of the collection flask. The material was liquefied by gently heating the distillation apparatus and top of the collection flask with a hot air gun. After the system reached equilibrium the temperature of the oil bath was increased to 250° C. For most crude oils the maximum temperature of the distillate vapor was between 140-162° C. After the vapor temperature decreased to below 100° C. the oil bath was removed and the system was allowed to come to room temperature. The distillate (vapors from the vacuum transfer and vacuum distillation steps that were condensed) was kept frozen using liquid nitrogen while the residue cooled to ambient temperature with stirring. It is important to stir the residue until it reaches ambient temperature so that the residue is homogeneous otherwise the vapor and condensate left in the distillation apparatus will collect on top of the residue and not mix with it. As the distillation flask reached ambient temperature acetone was used to rinse off any residual DPDM-400 silicon from the outside of the flask which was then wiped with several Kimwipes™.

After the distillation flask reached room temperature the liquid nitrogen was removed from the distillate flask (a type of collection vessel) and the distillate flask was allowed to reach about 0° C. and nitrogen was introduced into the distillation apparatus through the vacuum line and the distillation and distillate flasks were removed and capped with rubber septa. The flasks were taken to analytical balances, the septa were removed, the joints were wiped clean from any residual Krytox™ grease using Kimwipes™, and the weight of the distillation flask with residue and stir bar were recorded and the weight of the collection flask and distillate was also recorded. The mass of the residue and distillate were calculated as follows:

Residue=(weight of distillation flask+stir bar+oil) before distillation−(weight of distillation flask+stir bar+residue) after distillation

Distillate=(weight of collection flask+distillate)−(weight of the collection flask)

Such determinations are indeed types of analyses (producing residue and volatiles analysis results). Mass balance data and maximum distillation temperature at about 10 microns (0.01 mmHg) from the distillation of JS1-JS3, Desalt Out 1, Desalt Out 2, Minnelusa, and Dilbit are given below in Table 1 and the ¹H NMR data are given in Table 2.

TABLE 1
Mass balance and maximum vapor temperature distillation temperature data from
the distillation of JS1-JS3, Desalt Out 1, Desalt Out 2, Minnelusa, and Dilbit.

| | | | | | Max | Mass Fraction | Mass Fraction |
|---|---|---|---|---|---|---|---|
| | Oil (g) | Distillate (g) | Residue (g) | Mass Balance | Distillation ° C. | of Distillate | of Residue |
| JS1 | 42.7272 | 22.4674 | 19.9847 | 42.4521 | 145 | 0.526 | 0.468 |
| JS2 | 56.9429 | 40.2857 | 16.2942 | 56.5799 | 152 | 0.707 | 0.286 |
| JS3 | 47.2072 | 32.4784 | 14.3366 | 46.8150 | 156 | 0.688 | 0.304 |
| Desalt Out1 | 41.7513 | 14.6503 | 26.7236 | 41.3739 | 152 | 0.351 | 0.640 |
| Desalt Out2 | 40.3006 | 13.8965 | 26.1000 | 39.9965 | 145 | 0.345 | 0.648 |
| Minnelusa | 52.5085 | 25.0006 | 27.1634 | 52.1640 | 162 | 0.476 | 0.517 |
| Dilbit | 34.3235 | 11.9549 | 21.9316 | 33.8865 | 131 | 0.348 | 0.639 |

TABLE 2
¹H NMR data (volatiles analysis data) from the distillation of
JS1-JS3, Desalt Out 1, Desalt Out 2, Minnelusa, and Dilbit. The
saturates are integrated against the aromatics which are set
to one. The saturates are divided by 10 for convenience and multiplied
by the mass fraction of the distillate (condensed volatiles)
which gives the aromaticity of the distillate.

| | | | | Satu- | ×Mass | |
|---|---|---|---|---|---|---|
| | Satu- | Aro- | Ole- | rates ÷ | Fraction of | % Satu- |
| | rates | matics | fins | 10 | Distillate | rates |
| JS1 | 20.95 | 1 | Yes | 2.10 | 1.10 | 95.4 |
| JS2 | 22.81 | 1 | No | 2.28 | 1.61 | 95.8 |
| JS3 | 28.73 | 1 | No | 2.87 | 1.98 | 96.6 |
| Desalt Out1 | 21.25 | 1 | No | 2.13 | 0.75 | 95.5 |
| Desalt Out2 | 20.99 | 1 | No | 2.10 | 0.72 | 95.5 |
| Minnelusa | 28.30 | 1 | No | 2.83 | 1.35 | 96.6 |
| Dilbit | 39.02 | 1 | Yes | 3.90 | 1.36 | 97.5 |

Proton NMR was acquired with a Bruker DRX-400 instrument using 5 mm NMR tubes fitted with Teflon valves (Chemglass, CG-512; or New Era, NE-CAV5-M-170). A standard proton NMR pulse program was used with a delay time of 2 seconds. The distillates were added to the NMR tubes which were fitted with a custom sealed glass capillary filled with CD₂Cl₂. The spectra were referenced to the methylene chloride peak relative to TMS at 5.32 ppm. Integrations were carried out using the software and the bias and slopes were manually adjusted. The aromatic region was integrated from 6.5-9.3 ppm and set to one; the aliphatic region was integrated from -0.3-4.4 ppm. It was observed that JS1 contained significant olefin resonances between 4.8-6.1 ppm, and the Dilbit contained minor olefin resonances in the same region. Other information can be gathered from the ¹H NMR data such as an estimation of naphthenic species; and in the case of dilbit or synthetic crudes which have been diluted with light fractions such as C3-C6 diluents the methyl (CH₃, δ˜1.2) proton resonances are greater in intensity and integrated area relative to the methylene (CH₂, δ˜1.6) proton resonances which is not true for most dead crudes. Taking this into consideration with the fact that there are very little middle distillates and a large amount of residue, one may be able to determine if a crude is a synthetic crude or a heavy oil/bitumen diluent blend. In addition to other features indicated herein, NMR data can be used to calculate carbon chains, alpha carbons, cyclic naphthenic carbons, functional groups, quaternary carbons, olefins, X—H protons where X is the heteroatom O, N, or S, CH₃ carbons, CH₂ carbons, CH carbons, 2-dimensional NMR spectra correlating protons NMR spectra to carbon NMR spectra, 2-dimensional NMR spectra correlating carbon DEPT NMR spectra to proton NMR spectra

Analysis of the residues from JS1-JS3, Desalt Out 1, Desalt Out 2, Minnelusa, and Dilbit were carried out using the SAR-AD as described in (Boysen and Schabron 2013). Data (residue analysis results) from the SAR-AD are given in Table 3.

TABLE 3
SAR-AD data for residues JS1-JS3, Desalt Out 1, Desalt Out 2, Minnelusa, and Dilbit.

| | | | | | | | | | Coking | Total |
|---|---|---|---|---|---|---|---|---|---|---|
| | | Maltenes | | | Asphaltenes | | | Total ELSD | Index | Pericondensed |
| Sample | Detector | Saturates | Aromatics | Resins | CyC₆ | Toluene | CH₂Cl₂/MeOH | Asphaltenes | Cy₆/CH₂Cl₂ | Aromatics |
| QC PreRun | ELSD | 21.9 | 8.5 | 54.7 | 3.9 | 11.0 | 0.2 | 15.0 | 25.7 | 21.8 |
| | 500 nm | | 0.1 | 31.3 | 23.5 | 43.2 | 1.9 | | 12.7 | |
| | 700 nm | | 0.3 | 20.3 | 27.0 | 49.6 | 2.8 | | | |
| JS1 | ELSD | 36.3 | 8.5 | 42.2 | 2.7 | 10.0 | 0.3 | 13.0 | 10.5 | 18.1 |
| | 500 nm | | 0.1 | 28.2 | 21.1 | 47.0 | 3.6 | | 5.9 | |
| | 700 nm | | 0.2 | 18.0 | 23.8 | 53.4 | 4.6 | | | |
| JS2 | ELSD | 69.5 | 8.4 | 20.8 | 0.2 | 1.1 | 0.1 | 1.4 | 2.2 | 2.5 |
| | 500 nm | | | 46.0 | 12.6 | 35.0 | 6.4 | | 2.0 | |
| | 700 nm | | 1.5 | 28.7 | 16.2 | 42.9 | 10.8 | | | |
| JS3 | ELSD | 69.9 | 8.7 | 19.7 | 0.3 | 1.4 | 0.1 | 1.8 | 1.9 | 3.3 |
| | 500 nm | | 0.4 | 45.6 | 14.9 | 33.1 | 6.0 | | 2.5 | |
| | 700 nm | | 0.9 | 32.8 | 19.0 | 38.4 | 8.9 | | | |
| Desalt Out1 | ELSD | 29.4 | 10.0 | 48.2 | 2.0 | 9.9 | 0.6 | 12.5 | 3.7 | 17.5 |
| | 500 nm | | 0.1 | 28.7 | 17.0 | 48.2 | 6.1 | | 2.8 | |
| | 700 nm | | 0.2 | 18.4 | 19.1 | 54.5 | 7.8 | | | |
| Deslat Out2 | ELSD | 30.2 | 10.2 | 47.5 | 1.9 | 9.7 | 0.5 | 12.1 | 3.7 | 17.0 |
| | 500 nm | | 0.1 | 28.7 | 17.0 | 48.0 | 6.1 | | 2.8 | |
| | 700 nm | | 0.2 | 18.5 | 19.1 | 54.2 | 8.0 | | | |
| Minnelusa | ELSD | 42.4 | 7.3 | 38.8 | 1.5 | 9.5 | 0.6 | 11.6 | 2.5 | 14.0 |
| | 500 nm | | 0.2 | 17.5 | 18.1 | 55.3 | 8.9 | | 2.0 | |
| | 700 nm | | 0.1 | 27.4 | 16.0 | 49.9 | 6.7 | | | |
| Dilbit | ELSD | 19.1 | 8.5 | 57.7 | 3.9 | 10.7 | 0.2 | 14.7 | 24.3 | 21.7 |
| | 500 nm | | | 32.3 | 23.6 | 42.2 | 2.0 | | 12.0 | |
| | 700 nm | | 0.3 | 21.0 | 27.2 | 48.6 | 2.9 | | | |
| QC Post Run | ELSD | 22.0 | 8.7 | 54.1 | 4.3 | 10.6 | 0.3 | 15.2 | 14.3 | 22.2 |
| | 500 nm | | 0.1 | 31.4 | 24.6 | 42.1 | 1.8 | | 13.7 | |
| | 700 nm | | 0.3 | 20.6 | 28.2 | 48.5 | 2.4 | | | |

Improved analysis results such as a modified (or adjusted) colloidal instability index (CII_{M1}) values for crudes JS 1-JS3, Desalt Out 1, Desalt Out 2, Minnelusa, and Dilbit as shown in the tables) were calculated using the residue analysis results and the volatiles analysis results (e.g., the mass fraction of the distillate multiplied by the ¹H NMR aromaticity (the values were scaled to make values more similar to those of the standard CII, here we scaled them by diving the value by 10) multiplied by the mass fraction of the residue multiplied by the colloidal instability index, as follows):

[Mass Fraction Distillate×¹H NMR (saturates/aromatics)/10]×[Mass Fraction Residue×(CII)]

Indeed, this is but one way in which the hydrocarbon residue analysis results and the hydrocarbon volatiles analysis results may be used to generate improved results that characterize the untreated hydrocarbon (or hydrocarbon in it pre-treatment condition) with improved accuracy. The CII is calculated from the wt % of the SARA fraction which is the sum of asphaltenes and saturates divided by the sum of the resins and aromatics (CII=(saturates+asphaltenes)/(aromatics+resins)). Table 4 shows the values used to calculate the modified CII_{MI} (that applies to the pre-treatment oils) for the distillate/volatiles and residue of such oils—JS 1-JS3, Desalt Out 1, Desalt Out 2, Minnelusa, and Dilbit.

TABLE 4
Modified colloidal instability index using ¹H NMR data from the volatiles fraction and
the residue SAR-AD data of JS1-JS3, Desalt Out 1, Desalt Out 2, Minnelusa, and Dilbit.

| | Mass | Mass | Distillate Mass | | Residue Mass | |
|---|---|---|---|---|---|---|
| | Fraction of | Fraciton of | Fraction × ¹H NMR | | Fraction × | Modified |
| Oil | Distillate | Residue | Saturates/Aromatics/10 | SAR-AD CII | SAR-AD CII | CII (CII_{M1}) |
| JS1 | 0.53 | 0.47 | 1.10 | 0.97 | 0.45 | 0.50 |
| JS2 | 0.71 | 0.29 | 1.61 | 2.44 | 0.70 | 1.12 |
| JS3 | 0.69 | 0.30 | 1.98 | 2.53 | 0.77 | 1.52 |
| Desalt Out 1 | 0.35 | 0.64 | 0.75 | 0.72 | 0.46 | 0.34 |
| Desalt Out 2 | 0.34 | 0.65 | 0.72 | 0.73 | 0.47 | 0.34 |
| Minnelusa | 0.48 | 0.52 | 1.35 | 1.17 | 0.61 | 0.82 |
| Dilbit | 0.35 | 0.64 | 1.36 | 0.51 | 0.33 | 0.44 |

A vast combination or various alternate equations incorporating the data from the distillate (volatiles) and /or residue oils (e.g., from the residue analysis results and/or the volatiles analysis results) can be utilized to calculate various desired predictive or diagnostic parameters that are related to the properties of the hydrocarbon in its pre-treatment condition (i.e., before volatiles are removed from it). Many different equations can be used following the quantitative distillation and these are all part of the current invention. The improved analysis results can be used to improve predictability or control over one or more phenomena or process as described elsewhere herein.

The colloidal instability index in particular relates the problematic asphaltenes content and the asphaltene destabilizing saturates to the components in the oil which are good at dissolving asphaltenes—the aromatics and resins. Colloidal instability index values have been used as predictors for various oil phenomena such as heat exchanger fouling (Asomaning 2003), emulsions (2003 Sjoblom), blending and physical properties of asphalt (Gaestel Index, Oyekunle 2007), and other asphaltene related phenomena and processing and refining issues.

Accordingly, one aspect of the inventive technology may be described as method for analyzing a hydrocarbon that comprises volatiles, wherein the method comprises the steps of segregating the volatiles from the hydrocarbon without oxidizing the hydrocarbon; generating a hydrocarbon residue 20 and segregated hydrocarbon volatiles 21; and analyzing at least one of the hydrocarbon residue and the segregated hydrocarbon volatiles. The term volatiles (whether used without an adjective, or as a first volatiles portion/fraction, a second volatiles portion/fraction, a lighter volatiles portion/fraction, or a heavier volatiles portion/fraction) used herein are those components actually (eventually) evaporated (and typically segregated); they include at least substantially all components of the hydrocarbon that would evaporate if subjected to the particular evaporative analysis method (e.g., ELSD, used on the residue). Segregating more than those volatiles that would actually have been evaporated if subjected to a particular evaporative analysis method may be acceptable, in that it may not impair the accuracy of the results that apply to the untreated hydrocarbon (such results typically determined from results generated by an analysis of the residue and volatiles fractions). Volatiles of a hydrocarbon may be defined generally as those components that are actually evaporated during, e.g., the vacuum transfer and vacuum distillation steps; the first/lighter portion/fraction may be defined as being those components that are evaporated during a vacuum transfer step, perhaps in addition to having other boiling point related constraint(s); and the second/heavier portion/fraction may be those components that are evaporated in a vacuum distillation step, in addition to having other boiling point related constraints. Substantially all as used herein is at least that proportion of all volatiles below which results would be impacted to an unacceptable degree (in other words, there is a certain percentage of all of those components that would be evaporated if subjected to the evaporative analysis below which too much error is introduced into the results; removal of less than this percentage would result in results with an unacceptably high degree of error). Removal of substantially all indicates that enough of the evaporative components (i.e., the components that would be evaporated if subjected to the evaporative analysis) are removed so that results are good enough (i.e., have acceptably small error); substantially all is at least that percentage). Substantially all may be selected from the group consisting of at least 85%, at least 90%, at least 92.5%, at least 95%, at least 97.5%, at least 98%, at least 99%, and at least 99.5%. Note that any of the methods disclosed herein may involve a segregation of volatiles that is performed without cracking the hydrocarbon (including the residue); this maybe achieved by segregating under a vacuum (particularly those portions with higher boiling points, such as those at or above the hydrocarbon's cracking temperature).

Another aspect of the inventive technology may be described as a method of analyzing a hydrocarbon that comprises volatiles having a first volatiles portion and a second volatiles portion, wherein volatile components of the first volatiles portion have boiling points that are less than or equal to a first portion boiling point maximum (i.e., the greatest boiling point of the first portion); and wherein volatile components of the second volatiles portion have a second portion boiling point maximum that is higher (in value) than the first portion boiling point maximum. The method may comprise the steps of: segregating the first volatiles portion from the hydrocarbon; segregating the second volatiles portion of the hydrocarbon while the hydrocarbon is under a vacuum; wherein both said steps of segregating are performed without oxidizing the hydrocarbon (wherein such non-oxidation is effected by, e.g., placing the hydrocarbon under a vacuum); generating hydrocarbon residue and segregated hydrocarbon volatiles; and analyzing at least one of the hydrocarbon residue and the segregated hydrocarbon volatiles. The second volatiles portion may be generally described as that portion that is not amenable to segregation from the hydrocarbon via vacuum transferring (at the pressures used in such transfer, such transfer typically not involving the transfer of heat to hydrocarbon), and that instead requires heating and thus vacuum distillation (which typically does involve the addition of heat to the hydrocarbon) for segregation. Note that even where even a few molecules of either the second (or the first) portion of the volatiles is evaporated and segregated, the step of segregating the second (or the first) portion of the volatiles is said to have occurred.

Yet another aspect of the inventive technology may be described as a method of improving the results of conventional hydrocarbon analysis, the conventional hydrocarbon analysis effecting evaporation of volatiles present in a hydrocarbon during the analysis thereof (i.e., during analysis of the hydrocarbon), the method comprising the steps of: segregating the volatiles from a sample of the hydrocarbon while the sample (and generated residue) is under an inert atmosphere or oxygen free environment, thereby generating a hydrocarbon residue and segregated hydrocarbon volatiles; analyzing the hydrocarbon residue via the conventional hydrocarbon analysis to generate hydrocarbon residue analysis results, analyzing the segregated hydrocarbon volatiles to generate hydrocarbon volatiles analysis results, and using the hydrocarbon residue analysis results and the hydrocarbon volatiles analysis results to generate improved analysis results for the hydrocarbon in its pre-treatment condition. Such improved results are improved as compared to those results that conventional hydrocarbon analysis on the hydrocarbon in its pre-treatment condition would generate, in that the improved results account for the volatiles. In some, but not necessarily all embodiments, they may be a form of adjustment or modification of the results of the residue analysis (which includes modification of only one result of the residue analysis results, such as colloidal stability index). The improved results may be of a parameter such as, but not limited to, colloidal instability index; an example of how the results of the residue analysis (e.g., CII of the residue and mass fraction of the residue) may be adjusted or modified using results of the volatiles analysis (e.g., mass fraction of volatiles distillate and ¹H NMR aromaticity thereof) is elsewhere herein. Essentially, in certain of such embodiments, instead of simply using a desired, conventional technique (e.g., such as SAR-AD) to analyze a hydrocarbon (in its untreated condition (where volatiles have not been removed)), volatiles may be removed from the hydrocarbon to generate a residue and volatiles fraction. The conventional technique may be used to analyze the residue, and the results of analysis of the volatiles (and perhaps also some residue analysis results, such as mass or mass fraction) may be used to adjust the results of the analysis of the residue (e.g., to adjust CII) to generate results (e.g., modified CII) that characterize (i.e., provide any sort of information about) the pre-treatment hydrocarbon with sufficient (improved) accuracy. Note that the technique(s) used to analyze the residue and the volatiles may include (but certainly are not limited to) any that determine mass (e.g., mass balance thereof). Mass, mass fraction, weight, weight fraction, volume or volume fraction, etc., for the residue and volatiles are some of the analysis results that may be used (along with other results, of either the residue or volatiles analysis) to generate improved results that more accurately characterize the hydrocarbon in its pre-treatment condition. Results of the mass balance may be used either alone or in conjunction with other analysis results to generate improved results that characterize (provide any information about) the pre-treatment hydrocarbon with improved accuracy (e.g., relative to the use of conventional methods that are used on an untreated (i.e., without volatiles removed) hydrocarbon). Note that the inert or oxygen free environment may be effected by, for example, a vacuum.

An additional aspect of the inventive technology may be described as a method of analyzing a hydrocarbon that comprises volatiles, the volatiles comprising a first portion and a second portion, wherein volatile components of the first portion have boiling points that are less than or equal to a first portion boiling point maximum (a maximum boiling point of all volatiles that are evaporated during the vapor transfer step), and wherein volatile components of the second portion have a second portion boiling point maximum that is higher than the first portion boiling point maximum, the method comprising the steps of: vacuum transferring the first portion of the volatiles out of the hydrocarbon; and vacuum distilling the second portion of the volatiles out of the hydrocarbon. Note that, although condensing that which is transferred under vacuum during the vacuum transferring step is typically done, it may not be a required step in certain embodiments and the term vacuum transferring does not necessarily connote condensation of vapors. However, the term vacuum distillation does connote condensation. Vapors transferred out of the hydrocarbon via vacuum transferring typically are condensed and trapped (via a cooling bath) in a collection vessel (which may also hold distillate from generated via vacuum distillation).

Yet another independent inventive aspect of the inventive technology may be described as a method for analyzing a hydrocarbon that comprises volatiles, wherein the method comprises the steps of segregating the volatiles from the hydrocarbon (e.g., quantitatively segregating); generating a hydrocarbon residue and segregated hydrocarbon volatiles; and analyzing at least one of the hydrocarbon residue and the segregated hydrocarbon volatiles, wherein the step of segregating the volatiles from the hydrocarbon comprises the step of cooling the volatiles removed from the hydrocarbon (i.e., the volatiles, or a portion of the volatiles). Such cooling, as described elsewhere herein) may keep the volatiles in liquid form, prevent them from evaporating, and thus prevent them from migrating back to, and becoming part of, the hydrocarbon from which they were initially evaporated. Cooling of the volatiles may be achieved by cooling a collection vessel that contains the volatiles, perhaps with a cooling bath (e.g., a liquid nitrogen cooling bath). However, other manners of cooling known in the prior art may also be used.

Note that at least one embodiment of the inventive technology may be described as a method comprising the steps of: establishing a hydrocarbon into a distillation unit comprising:a sealed system that prevents loss of volatiles through the vacuum pump; a cooled collection vessel to trap the volatiles of the hydrocarbon; a heating system that causes the hydrocarbon to reach a hydrocarbon (e.g., oil) distillation temperature below 340° C.; and an inert atmosphere to prevent oxidation of the hydrocarbon (such as of its various components).

Particular embodiments of these aspects may be described as follows, in addition to being as described in remaining portions of this specification.

In preferred embodiments, the hydrocarbon is a liquid hydrocarbon; at times, albeit atypically, a solvent may be added to a hydrocarbon, with the resulting solution still termed a hydrocarbon.

In those embodiment articulated relative to a first portion boiling point maximum and a second portion boiling point maximum, the first portion boiling point maximum may be less than about 160 degs C., less than 140 degs. C., less than 120-130 degs C. (e.g., less than about 125 or 120 degs C.), or indeed it may have other values while the second portion boiling point maximum may be any one (or more) of the following: between approximately 120 and 540 deg. C., between approximately 130 and 500 deg. C., between approximately 140 and 480 deg. C., between approximately 160 and 440 deg. C., and less than approximately 540 deg. C. All such values are corrected to atmospheric pressure. As used herein, approximately suggests within 10% (of the indicated value) below and 10% above the indicated value. Note further that any embodiment articulated relative to first and second volatile portions may include the step of condensing the first and/or second portion(s). And while certainly not necessarily required, where indicated in the claims, certain embodiments may be performed in the order in which they appear.

As mentioned, volatiles that have been removed from the hydrocarbon should be kept from somehow returning to the hydrocarbon and again becoming a component of the hydrocarbon. Such segregation of the volatiles (or any portion thereof) may be achieved, in certain embodiments, by cooling the volatiles (after their removal from the hydrocarbon). This may be accomplished, in particular embodiments, by cooling the volatiles with a cooling bath (e.g., a liquid nitrogen bath, a dry ice cooling bath, a dry ice acetone cooling bath, a salt and ice and acetone cooling bath, a slurry made with solvents and dry ice cooling bath, a slurry made with solvents and liquid nitrogen cooling bath, a liquid helium cooling bath, and a liquid helium solvent slurry cooling bath, e.g.) or an electric chiller (as but a few examples). Typically this is done when the volatiles are in a collection vessel. Such cooling may be achieved, in at least one embodiment, via a liquid nitrogen cooling bath. Cooling may also be achieved by cooling the distillation condenser column but this, at times, can solidify hydrocarbon portions, especially waxes, inhibiting further separation.

The hydrocarbon may be of the following type: oil, crude oil, biofuel, petroleum oil, shale oil, coal-derived oil, synthetic oil, vegetable oil, nut oil, fossil oil, biomass derived oil, oil constituent, asphalt binder, dilbit, opportunity crude oil, extra heavy oil, high TAN crude, solvent diluted oil, undiluted oil, oil with additive, oil from environmental release, pollutant oil, lubrication oil, recycled oil, asphalt material, tar sands bitumen, feed oil, asphalt, coal liquid, bitumen, crude oil, light crude oil, medium crude oil, cracked hydrocarbon, partially cracked hydrocarbon, medium crude oil, heavy crude oil, extra heavy crude oil, synthetic crude oil, blended crude oil, food grade oil, and oil-base cosmetics. The hydrocarbon may also be a very low asphaltene content oil that is not amenable to conventional Heithaus or other flocculation titration methods.

In particular embodiments, the step of analyzing at least one of the hydrocarbon residue and the hydrocarbon volatiles (which are typically segregated from the residue) comprises the step of analyzing the hydrocarbon residue, analyzing the (segregated) hydrocarbon volatiles, or analyzing both the hydrocarbon residue and the (segregated) hydrocarbon volatiles. The step of analyzing the hydrocarbon residue may comprise the step of analyzing with an evaporative analysis, which is an analysis that would evaporate at least a majority portion of the volatiles (actually evaporated) if the volatiles were subjected to that analysis (whether as segregated or as a part of a hydrocarbon); the step of segregating the volatiles may comprise the step of segregating at least substantially all of those components of the hydrocarbon that would evaporate if subjected to the evaporative analysis. In those embodiments that include the step of segregating (from the hydrocarbon) the volatiles (typically this step is indeed performed), such step may comprise segregating at least substantially all of those components of the hydrocarbon that would evaporate if subjected to the evaporative analysis. As such, the volatiles may be described, at least in part, for certain embodiments, as including at least those components of the hydrocarbon that would evaporate if subjected a residue analysis that is evaporative in nature.

In embodiments that include the step of segregating the volatiles from the hydrocarbon (which may be done without cracking the hydrocarbon), such step may comprise segregating at least a portion of the volatiles under a vacuum, which itself may comprise segregating at least a heavier portion of the volatiles under the vacuum (e.g., by vacuum distilling); certain embodiments that include the step of segregating at least a portion of the volatiles under a vacuum may include the step of segregating the volatiles under a vacuum (which may change throughout the process). The step of segregating the volatiles under a vacuum may also comprise the step of vacuum transferring a lighter portion of the volatiles and vacuum distilling a heavier portion of the volatiles. Those embodiments articulated as having a first volatiles portion and a second volatiles portion (where such portions may be defined according to maximum boiling point of the first portion and maximum boiling point of the second portion) may include the step of segregating the first volatiles portion from the hydrocarbon (e.g., via vacuum transferring the first volatiles portion from the hydrocarbon) and/or segregating the second volatiles portion of the hydrocarbon while the hydrocarbon is under a vacuum (e.g., by vacuum distilling the hydrocarbon). Note that the vacuums may, but need not, be different. In embodiments that include the step of segregating the volatiles from a (sample of a) hydrocarbon (e.g., by physically separating therefrom), such step may comprise the step of segregating the volatiles under vacuum (e.g., typically via vacuum transfer and vacuum distillation). Segregation may generate segregated volatiles, and a residue (i.e., that which remains of the hydrocarbon after volatiles are removed therefrom). Particular embodiments of methods that comprise the step of generating a hydrocarbon residue and segregated hydrocarbon volatiles may achieve such residue and volatiles via, for example, a vacuum process(es), such as vacuum transfer and vacuum distillation.

In those embodiments that include the step of analyzing at least one of the hydrocarbon residue and the segregated hydrocarbon volatiles, such step may comprise the step of analyzing the hydrocarbon residue and/or analyzing the segregated hydrocarbon volatiles. The step of analyzing the hydrocarbon residue may comprise the step of analyzing using a technique (e.g., a conventional technique) selected from the group of techniques consisting of: evaporative light scattering detector (ELSD), evaporative method, charged aerosol detector (CAD), on-column precipitation method, re-dissolution method, Asphaltene Determinator, Waxphaltene Determinator, Saturates Aromatics Resins-Asphaltene Determinator (SAR-AD), adsorbent column separations of saturates, aromatics, resins, and asphaltenes, nuclear magnetic resonance (NMR) spectroscopy, titration, acid-base titration, gas chromatography, one-dimensional gas chromatography, two-dimensional gas chromatography, multi-dimensional gas chromatography, gas chromatography/mass spectrometry (GC/MS), high performance liquid chromatography (HPLC), ion exchange chromatography, fluorescence spectroscopy, turbidimetric spectroscopy, ultraviolet (UV) spectroscopy, ultraviolet visible (UV-Vis) spectroscopy, infrared (IR) spectroscopy, Fourier Transform infrared (FTIR) spectroscopy, attenuated total reflectance infrared spectroscopy (ATR-IR), Raman spectroscopy, near infrared spectroscopy (NIR), acousto-optic tunable filter near infrared (AOTF-NIR) spectroscopy, Fourier Transform Raman spectroscopy, high resolution Fourier transform ion cyclotron resonance mass spectrometry (FTICR/MS), optical microscopy, X-ray microscopy, X-ray fluorescence spectroscopy, refractive index, supercritical fluid chromatography, supercritical fluid extraction, electrical resistivity, differential scanning calorimetry (DSC), thermogravimetric analysis (TGA), and flocculation titration (as but a few examples). In any residue analysis (indeed, perhaps also in any of the volatiles analysis), there may be little (or minimal) to no volatiles loss. Analyzing residue may include the step of minimizing volatiles loss, or effecting acceptably low volatiles loss (such that the resulting improved results, which characterize the pre-treatment or untreated hydrocarbon, will be of adequate accuracy); residue analysis may be performed with oxidizing the residue.

The step of analyzing the segregated hydrocarbon volatiles (e.g., a volatiles distillate) may comprise the step of analyzing using a technique selected from the group of techniques consisting of: NMR spectroscopy, titration, acid-base titration, gas chromatography, one-dimensional gas chromatography, two-dimensional gas chromatography, multi-dimensional gas chromatography, GC/MS, ATR-IR, HPLC, UV spectroscopy, UV-vis spectroscopy, IR spectroscopy, FT-IR spectroscopy, NIR spectroscopy, AOTF-NIR spectroscopy, FTICR/MS, optical fluorescence spectroscopy, turbidimetric spectroscopy, x-ray fluorescence spectroscopy, refractive index, supercritical fluid chromatography, supercritical extraction, mass balance or other “weighing” technique to determine mass or weight, and electrical resistivity. The analyses of the residue and the volatiles may involve different techniques (which is typical), or might involve the same technique. Any method may further comprise the step of comparing results generated by the step of analyzing the hydrocarbon residue with results generated by the step of analyzing the segregated hydrocarbon volatiles (e.g., as where it is desired to determine a relative amount of acid in the residue and acid in the volatiles fractions); in such case, the techniques used in the residue and the volatiles analysis may (but again, need not necessarily) be the same. Note that each of the steps of vacuum transferring the first portion of the volatiles out of the hydrocarbon and the step of vacuum distilling the second portion of the volatiles out of the hydrocarbon may comprise the step of condensing such respective (first or second) portion of the volatiles. Performance of the steps of vacuum transferring and vacuum distilling may together generate a hydrocarbon residue and segregated hydrocarbon volatiles.

Particular embodiments of the inventive technology may relate to use of analysis results to improve results that would have been obtained through the use strictly of conventional analyses. Such improved results (whether in the form of an adjustment or modification of one or more of the results of the residue analysis or not) may be achieved by using the results of the (segregated) volatiles analysis to adjust the results of the residue analysis. More particularly, as described elsewhere herein, residue and volatiles may be weighed to generate certain mass-related results, more sophisticated analyses described herein may be used to generate results that indicate how much (e.g., fractional amount (mass or volume) of each the residue and the volatiles are found in saturates, aromatics, resins and asphaltenes (typically volatiles are found to include primarily saturates and aromatics). Residue analysis results (e.g., of CII, for example) may be adjusted using the volatiles results so that such former residue results now are characteristic of the hydrocarbon in its pre-treatment condition. Accordingly, at least one embodiment of the inventive technology that includes the step of analyzing at least one of the hydrocarbon residue and the segregated hydrocarbon volatiles may comprise the step of analyzing the hydrocarbon residue and analyzing the segregated hydrocarbon volatiles. In such (and other) embodiments, the step of analyzing the hydrocarbon residue may comprise the step of generating hydrocarbon residue analysis results and the step of analyzing the segregated hydrocarbon volatiles may comprise the step of generating volatiles analysis results. The hydrocarbon residue analysis results and the hydrocarbon volatiles analysis results may be used to generate improved analysis results, for the hydrocarbon in its pre-treatment condition (recall that in particular applications, without the inventive technology: (a) any evaporative residue analysis would still evaporate some volatiles left in the residue, resulting in error in the residue analysis and thus an inability to properly adjust such results to account for the volatiles and an inability therefore to derive sufficiently accurate results for the hydrocarbon in its pretreatment condition; or (b) simply, the (evaporative) technique used in the residue analysis, as applied to the pre-treatment condition hydrocarbon, would yield inaccurate results for such hydrocarbon). The improved analysis results, in accounting for the volatiles, may be used to improve predictability or control of one or more phenomena, wherein the phenomena are selected from the group consisting of emulsion-related effects, emulsion formation, breaking of emulsions, emulsion formulation, fouling generally, heat exchanger fouling, distillation tower fouling, catalyst fouling, catalyst efficiency, settling, asphaltene deposition, asphaltene precipitation, sediment formation and asphaltene adsorption. The improved analysis results may be used to improve control of at least one process, regardless of what that process may be.

Note that generally, the term hydrocarbon (which includes a sample of a hydrocarbon of course) may refer to the hydrocarbonaceous material in its pre-treatment condition, its post-treatment condition (after volatiles are removed therefrom), or any intermediate condition (i.e., its condition during the volatiles removal process). A pre-treatment hydrocarbon refers specifically to a hydrocarbon in its condition before any volatiles are removed from it. Indeed, the improved results better characterize the hydrocarbon in its pre-treatment, or untreated, condition. A hydrocarbon residue is the hydrocarbon after the volatiles, or at least a portion thereof, have been removed from it.

One beneficial aspect of certain embodiments of the inventive technology may be removal of the volatiles from a hydrocarbon without cracking the hydrocarbon. In certain embodiments, this is achieved by heating the hydrocarbon such that the highest temperature of the hydrocarbon does not meet or exceed a cracking temperature (e.g., 340 deg. C.). Accordingly, the step of segregating the second volatiles portion of the hydrocarbon while the hydrocarbon is under a vacuum may be performed without cracking the hydrocarbon, the step of segregating the volatiles from the hydrocarbon may be performed without cracking the hydrocarbon, and/or each of the steps of vacuum distilling and vacuum transferring does not effect cracking of the hydrocarbon.

A goal of certain embodiments of the inventive technology may be to prevent oxidation of the hydrocarbon. This may be achieved by performing the step of segregating the volatiles from the hydrocarbon while the hydrocarbon (including residue) is under a vacuum (as more particularly described elsewhere herein), which may achieve an inert atmosphere.

As mentioned, there may be several different applications of one or more of the inventive technologies articulated and disclosed herein. For example, certain embodiments may seek to determine relative concentrations of acids in the segregated hydrocarbon volatiles and hydrocarbon residue (note that embodiments may achieve this benefit even where the residue analysis is not evaporative). One application may seek to determine whether olefins are present in the hydrocarbon. In another, the inventive technology may be used to determine the stability of a pyrolytic process (including but not limited to thermal cracking, catalytic cracking, hydrocracking, coking and thermal upgrading) to which the hydrocarbon has been subjected. Perhaps more generally, an application may intend to characterize the hydrocarbon (including but not limited to identifying the hydrocarbon as being a hydrocarbon selected from the group consisting of: bitumen, heavy oil blended with a light diluent, and heavy oil or bitumen blended with a light oil, medium oil, extra heavy oil, a blend thereof, and heavy oil or bitumen blended with other processed streams). Certain embodiments may be used to monitor a thermal cracking and upgrading process to which the hydrocarbon has been subjected; others may seek to determine relative concentrations of acids in the segregated hydrocarbon volatiles and hydrocarbon residue. Improved results for the hydrocarbon in its pre-treatment condition may be used to increase distillate yield as compared to a distillate yield that would result from use of results that a conventional hydrocarbon analysis would generate for the pre-treatment condition hydrocarbon. Improved results may be used to improve predictability or control of one or more phenomena (as but a few examples): emulsion-related effects, emulsion formation, breaking of emulsions, fouling generally, heat exchanger fouling, distillation tower fouling, catalyst fouling, catalyst efficiency, settling, asphaltene deposition, asphaltene precipitation, sediment formation and asphaltene adsorption. Generally, the improved results may be used to improve control of at least one process.

It is of note that the applicant also claims as within the ambit of the inventive technology the following: a refinery or apparatus in which any of the claimed methods is performed; a refinery that processes hydrocarbons based on analysis results generated, at least in part, upon performance of any of the claimed methods; a product produced by a process that is based on analysis results generated, at least in part, upon performance of any of the claimed methods; a supplemental amount of hydrocarbon end product per a given unprocessed hydrocarbon amount, the supplemental amount produced, at least in part, due to information gained upon performance of any of the claimed methods (where the supplemental amount may be generated due to improvements in efficiency afforded by the inventive technology); a refinery that produces a supplemental amount of oil whose production is realized, at least in part, due to performance of any of the claimed methods; a supplemental amount of hydrocarbon end product per a given pollutant emitted during processing of a input hydrocarbon, the supplemental amount produced, at least in part, due to information gained upon performance of any of the claimed methods; unprocessed hydrocarbon that is eventually processed using information gained by performance any of the claimed methods; a monetary amount associated with a decrease in refinery greenhouse emissions, the decrease due, at least in part, to production efficiency gains realized by use of results of information determined using any of the claimed methods; a method as described in any of the claims that claim a monetary amount, where that monetary amount is associated with a traded, cap and trade emissions credit; and a method as described in any of the claimed methods wherein the method is a method selected from the group consisting of coking onset estimation method, oil processing method; oil fractionating method, oil production method, reservoir fouling related method, pipeline fouling related method, hydrotreating method, heat exchanger fouling method, catalytic upgrading method, distillation method, vacuum distillation method, atmospheric distillation method, visbreaking method, blending method, asphalt formation method, emulsion-related method, emulsion formation method, emulsion breaking method, emulsion formulation method, oil subfraction measurement method, asphalt extraction method, and asphaltene content of oil measurement method. The inventive technology also includes controlling, designing or monitoring processing of a hydrocarbon through use of information generated from performance of any of the claimed methods. It further includes a method as described in any of the claimed methods wherein analysis results generated through use of the method are used to further a processing related goal selected from the group consisting of: increasing distillate yield and quality; displacing high-sulfur fuel oil; boosting propylene output; mitigating fouling and corrosion; and reducing carbon footprint.

One apparatus type aspect of the inventive technology may be described as an apparatus 1 for analyzing a hydrocarbon so as to generate results that are more accurate than are results generated using conventional analysis, the hydrocarbon comprising volatiles having a first volatiles portion and a second volatiles portion, wherein volatile components of the first volatiles portion have boiling points that are less than or equal to a first portion boiling point maximum; and wherein volatile components of the second volatiles portion have a second portion boiling point maximum that is higher than the first portion boiling point maximum, the apparatus comprising: closed distillation apparatus 2 configured to segregate the volatiles from the hydrocarbon to generate a hydrocarbon residue and segregated hydrocarbon volatiles; hydrocarbon residue analysis componentry 3 configured to analyze the hydrocarbon residue and generate hydrocarbon residue analysis results for the hydrocarbon residue; volatiles analysis componentry 4 configured to analyze the segregated hydrocarbon volatiles and generate hydrocarbon volatiles analysis results for the segregated hydrocarbon volatiles; and computer componentry 5 (e.g., a microprocessor) configured to use the hydrocarbon residue analysis results and the hydrocarbon residue analysis results to generate improved results (in the form of a modified colloidal stability index results, asphaltene stability index results, Gaestel index results, and resins to asphaltene ratio results, as but a few examples) that more accurately characterize the hydrocarbon in its pre-treatment condition.

Another apparatus type aspect of the inventive technology may be described as an apparatus for analyzing a hydrocarbon so as to generate results that are more accurate than are results generated using conventional analysis, the hydrocarbon comprising volatiles, the apparatus comprising: vacuum apparatus (e.g., vacuum distillation apparatus 2) configured to segregate the volatiles from the hydrocarbon to generate a hydrocarbon residue and segregated hydrocarbon volatiles, wherein the vacuum apparatus comprises volatiles cooling apparatus 10 (a cooling apparatus to collect volatiles); hydrocarbon residue analysis componentry 3 configured to analyze the hydrocarbon residue and generate hydrocarbon residue analysis results for the hydrocarbon residue; volatiles analysis componentry 4 configured to analyze the segregated hydrocarbon volatiles and generate hydrocarbon volatiles analysis results for the segregated hydrocarbon volatiles; and computer componentry 5 configured to generate, from the hydrocarbon volatiles analysis results and the hydrocarbon residue analysis results, improved results that characterize the hydrocarbon in its pre-treatment condition. The volatiles cooling apparatus may comprise a cooling bath apparatus 11 (a liquid nitrogen cooling bath apparatus, as but one example), a chiller, and/or a cryogenic (fluid or solid)-based cooling apparatus. The vacuum apparatus may prevent oxidation of the hydrocarbon (including residue), among having other effects.

The closed distillation apparatus (closed in that it can hold a negative or vacuum pressure, such as the low pressures indicated herein) may comprise an apparatus selected from the group consisting of: vacuum jacketed distillation apparatus, and Vigreux vacuum jacketed distillation apparatus 12 (as but a few of many examples). The closed distillation apparatus may comprise a distillation flask 15, a condenser 16, a vacuum system 17, and a collection flask 18, a distillation flask heating system 19, and collection flask cooling system 10. The vacuum system of the closed distillation apparatus may effect non-oxidation of the hydrocarbon (which includes the generated residue). It may also allow for boiling of volatile components that have boiling points that are higher than the hydrocarbon's cracking temperature without actually causing any cracking of that hydrocarbon (including its residue). The hydrocarbon residue analysis componentry may comprise componentry configured to perform an analysis using any of the following techniques: ELSD detector, evaporative method, on-column precipitation method, re-dissolution method, Asphaltene Determinator, Waxphaltene Determinator, Saturates Aromatics Resins-Asphaltene Determinator, nuclear magnetic resonance spectroscopy, one-dimensional gas chromatography, two-dimensional gas chromatography, multi-dimensional gas chromatography, gas chromatography/mass spectrometry, high performance liquid chromatography fluorescence spectroscopy, turbidimetric spectroscopy, ultraviolet spectroscopy, infrared spectroscopy, attenuated total reflectance infrared spectroscopy, Raman spectroscopy, Fourier transfer Raman spectroscopy, near infrared spectroscopy, acousto-optic tunable filter near infrared spectroscopy, high resolution Fourier transform ion cyclotron resonance mass spectrometry (FTICR/MS), optical microscopy, X-ray microscopy, X-ray fluorescence spectroscopy, refractive index, supercritical fluid chromatography, supercritical fluid extraction, electrical resistivity, differential scanning calorimetry (DSC), thermogravimetric analysis (TGA), and flocculation titration. The volatiles analysis componentry comprises componentry configured to perform an analysis using a technique selected from the group consisting of: NMR spectroscopy, gas chromatography, HPLC, UV spectroscopy, IR spectroscopy, optical fluorescence spectroscopy, turbidimetric spectroscopy, x-ray fluorescence spectroscopy, refractive index, supercritical fluid chromatography, supercritical extraction, and electrical resistivity.

Note that additional aspects of the inventive technology may be described as follows: a refinery in which any of the claimed apparatus is located; a refinery that processes hydrocarbons based on analysis results generated, at least in part, upon use of any of the claimed apparatus; a product produced by a process that is based on analysis results generated, at least in part, upon use of any of the claimed apparatus; a supplemental amount of hydrocarbon end product per a given unprocessed hydrocarbon amount, the supplemental amount produced, at least in part, due to information gained upon use of any of the claimed apparatus; a refinery that produces a supplemental amount of oil whose production is realized, at least in part, upon use of any of the claimed apparatus; a supplemental amount of hydrocarbon end product per a given pollutant emitted during processing of a input hydrocarbon, the supplemental amount produced, at least in part, due to information gained upon use of any of the claimed apparatus; unprocessed hydrocarbon that is eventually processed using information gained use of any of the claimed apparatus; a monetary amount associated with a decrease in refinery greenhouse emissions, the decrease due, at least in part, to production efficiency gains realized by use of results of information determined using any of the claimed apparatus (e.g., where the monetary amount is associated with a traded, cap and trade emissions credit); an apparatus as described in any apparatus claim wherein the apparatus is selected from the group consisting of coking onset estimation apparatus, oil processing apparatus; oil fractionating apparatus, oil production apparatus, reservoir fouling related apparatus, pipeline fouling related apparatus, hydrotreating apparatus, distillation apparatus, vacuum distillation apparatus, atmospheric distillation apparatus, visbreaking apparatus, blending apparatus, asphalt formation apparatus, emulsion-related apparatus, emulsion formation apparatus, emulsion breaking apparatus, emulsion formulation apparatus, exchanger fouling apparatus, catalytic upgrading apparatus, asphalt extraction apparatus, oil subfraction measurement apparatus, and asphaltene content of oil measurement method; and an apparatus as described in any of the claimed apparatus wherein analysis results generated through use of the apparatus are used to further a processing related goal selected from the group consisting of: increasing distillate yield and quality; displacing high-sulfur fuel oil; boosting propylene output; mitigating fouling and corrosion; and reducing carbon footprint. It is of note that the inventive technology may also include the step of controlling, designing or monitoring processing of a hydrocarbon through use of information generated through use of any of the claimed apparatus. The results of the analysis can be used to predict properties of blends. The results of the analysis can be used to predict properties of blends based on the analysis of hydrocarbons prior to blending. The results of the analysis can be used to compare process efficiencies and product hydrocarbon properties under different processing conditions.

Additional Information: As can be easily understood from the foregoing, the basic concepts of the present invention may be embodied in a variety of ways. It involves both hydrocarbon constituent separation and/or analysis techniques as well as devices to accomplish the appropriate separation and/or analysis. In this application, the separation and/or analysis techniques are disclosed as part of the results shown to be achieved by the various devices described and as steps which are inherent to utilization. They are simply the natural result of utilizing the devices as intended and described. In addition, while some devices are disclosed, it should be understood that these not only accomplish certain methods but also can be varied in a number of ways. Importantly, as to all of the foregoing, all of these facets should be understood to be encompassed by this disclosure.

The discussion included in this application is intended to serve as a basic description. The reader should be aware that the specific discussion may not explicitly describe all embodiments possible; many alternatives are implicit. It also may not fully explain the generic nature of the invention and may not explicitly show how each feature or element can actually be representative of a broader function or of a great variety of alternative or equivalent elements. Again, these are implicitly included in this disclosure. Where the invention is described in device-oriented terminology, each element of the device implicitly performs a function. Apparatus claims may not only be included for the device described, but also method or process claims may be included to address the functions the invention and each element performs. Neither the description nor the terminology is intended to limit the scope of the claims that will be included in any subsequent patent application.

It should also be understood that a variety of changes may be made without departing from the essence of the invention. Such changes are also implicitly included in the description. They still fall within the scope of this invention. A broad disclosure encompassing both the explicit embodiment(s) shown, the great variety of implicit alternative embodiments, and the broad methods or processes and the like are encompassed by this disclosure and may be relied upon when drafting the claims for any subsequent patent application. It should be understood that such language changes and broader or more detailed claiming may be accomplished at a later date (such as by any required deadline) or in the event the applicant subsequently seeks a patent filing based on this filing. With this understanding, the reader should be aware that this disclosure is to be understood to support any subsequently filed patent application that may seek examination of as broad a base of claims as deemed within the applicant's right and may be designed to yield a patent covering numerous aspects of the invention both independently and as an overall system.

Further, each of the various elements of the invention and claims may also be achieved in a variety of manners. Additionally, when used or implied, an element is to be understood as encompassing individual as well as plural structures that may or may not be physically connected. This disclosure should be understood to encompass each such variation, be it a variation of an embodiment of any apparatus embodiment, a method or process embodiment, or even merely a variation of any element of these. Particularly, it should be understood that as the disclosure relates to elements of the invention, the words for each element may be expressed by equivalent apparatus terms or method terms—even if only the function or result is the same. Such equivalent, broader, or even more generic terms should be considered to be encompassed in the description of each element or action. Such terms can be substituted where desired to make explicit the implicitly broad coverage to which this invention is entitled. As but one example, it should be understood that all actions may be expressed as a means for taking that action or as an element which causes that action. Similarly, each physical element disclosed should be understood to encompass a disclosure of the action which that physical element facilitates. Regarding this last aspect, as but one example, the disclosure of a “analyzer” should be understood to encompass disclosure of the act of “analyzing”—whether explicitly discussed or not—and, conversely, were there effectively disclosure of the act of “analyzing”, such a disclosure should be understood to encompass disclosure of an “analyzer” and even a “means for analyzing”. Such changes and alternative terms are to be understood to be explicitly included in the description. Further, each such means (whether explicitly so described or not) should be understood as encompassing all elements that can perform the given function, and all descriptions of elements that perform a described function should be understood as a non-limiting example of means for performing that function.

Any patents, publications, or other references mentioned in this application for patent are hereby incorporated by reference. Any priority case(s) claimed by this application is hereby appended and hereby incorporated by reference. In addition, as to each term used it should be understood that unless its utilization in this application is inconsistent with a broadly supporting interpretation, common dictionary definitions should be understood as incorporated for each term and all definitions, alternative terms, and synonyms such as contained in the Random House Webster's Unabridged Dictionary, second edition are hereby incorporated by reference. Finally, all references listed in the list of References To Be Incorporated By Reference In Accordance With The Patent Application or other information statement filed with the application are hereby appended and hereby incorporated by reference; however, as to each of the above, to the extent that such information or statements incorporated by reference might be considered inconsistent with the patenting of this/these invention(s) such statements are expressly not to be considered as made by the applicant(s).

### LIST OF REFERENCES TO INCORPORATE INTO THIS APPLICATION, BY REFERENCE

Andersen, S. I. and E. II Stenby. 1996. Thermodynamics of Asphaltene Precipitation and Dissolution Investigation of Temperature and Solvent Effects. Fuel. Set. Technol. Int. 14: 261-287
Aske, N., II. Kallevik, and J. Sjoblom, 2001. Determination of Saturate, Aromatic, Resin and Asphaltenic (SARA) Components in Crude Oils by Means of Infrared and Near-Infrared Spectroscopy. Energy and Fuels. 15: 1304-1312.
Asomaning. S., 2003, Test Methods for Determining Asphaltene Stability in Crude Oils. Pet. Set and Tech. 21:581-590.
ASTM D86-12. 2012. Standard Test Method for Distillation of Petroleum Products at Atmospheric Pressure.
ASTM D2007-11, 2011. Standard Test Method for Characteristic Groups in Rubber Extender and Processing Oils and Other Petroleum-Derived Oils by the Clay-Gel Absorption Chromatographic Method.
ASTM D2887-13, 2013. Standard Test Method for Boling Range Distribution of Petroleum Fractions by Gas Chromatography.
ASTM D2892-13. 2013. Standard Test Method for Distillation of Crude Petroleum (15-Theoretical Plate Column).
ASTM D3279-97, 1997, Standard Test Method for n-Heptane Insolubles.
ASTM D3710-95 (Reapproved 2009), 2009. Standard Test Method for Boiling Range Distribution of Gasoline and Gasoline Fractions by Gas Chromatography.
ASTM D4124-01, 2001, Standard Test Methods for Separation of Asphalt into Four Fractions.
ASTM D5236-13, 2013, Standard Test Method for Distillation of Heavy Hydrocarbon Mixtures (Vacuum Potstill Method).
ASTM D7169-11. 2011, Standard Test Method for Boiling Point Distribution of Samples with Residues Sjuch as Crude Oils and Atmospheric and Vacuum Residues by High Temperature Gas Chromatography.
Balabin, R. M., and R. Z. J. Safieva. 2007, Capabilities of Near Infrared Spectroscopy for the Determination of Petroleum Macromolecule Content in Aromatic Solutions. J. Near Infrared Spectrosc. 15: 343-349.
Bombardelli, C. Assis. L. M., and H. J. Kalinowski, 2010. Tracking Aromaticity Changes in Heavy Hydrocarbon Processing by Monitoring Changes in Electrical Resistivity. Fuel, 3730-3734.
Boysen, R. B., and J. F. Schabron, 2013. The Automated Asphaltene Determinaior Coupled with Saturates, Aromatics. Resins Separation for Petroleum Residua Characterization. Energy and Fuels, 27: 4654-4661.
Buenrostro-Gonzalez, E., Groenzin. H., Lira-Galeana, G, and O. C. Mullins. 2001. The Overriding Chemical Principles that Define Asphaltenes. Energy Fuels. 15: 972-978.
Clutter, D. R., Petrakis. L., Stenger, R. L. Jr., and R. K. Jensen. 1972. Nuclear Magnetic Resonance Spectrometry of Petroleum Fractions. Carbon-13 and Proton Nuclear Magnetic Resonance Characterizations in Terms of Average Molecular Parameters. Anal. Chem., 44: 1395-1405.
Corbett, L. W., 1969. Composition of asphalt Based on Generic Fractionation. Using Solvent Deasphaltening. Elution-Adsorption Chromatography, and Densimelric Characterization. Analytical Chemistry, 41 (4): 576-579.
de Peinder, P. 2009, Characterization and Classification of Crude Oils Using a Combination of Spectroscopy and Chemometrics. Ph.D. Dissertation, University of Utrecht.
Fan, T., and J. S. Buckley. 2002. Rapid and Accurate Sara Analysis of Medium Gravity Crude Oils. Energy and Fuels, 16: 1571-1575.
Fossen, M., Kallevik. H., Knudsen. K. D., and J. Sjoblom. 2011. Asphaltenes Precipitated by a Two-Step Precipitation Procedure. 2. Physical and Chemical Characteristics. Energy Fuels, 25: 3552-3567.
Grizzle, P. L., and D. M. Sablotny. 1986, Automated Liquid Chromatographic Compound Class Group-Type Separation of Crude Oils and Bitumens Using Chemically Bonded Aminosilane. Analytical Chemistry, 58: 2389-2396.
Hayes, P. C. Jr., and S. D. Anderson, 1986, Hydrocarbon Group Type Analyzer System for the Rapid Determination of Saturates, Olefins, and Aromatics in Hydrocarbon Distillate Products. Anal. Chem, 58: 2384-2388.
Jewel, D. M., Weber, J. H., Bunger, J. W., Plancher. H., and D. R. Latham, 1972, Ion-Exchange, Coordination, and Adsorption Chromatographic Separation of Heavy-End Petroleum Distillates. Analytical Chemistry, 44: 1391-1395.
Jiang, C, S. R. Larter. K.J. Noke, and L. R. Snowdon. 2008. TLC-FID (latroscan) Analysis of Heavy Oil and Tar Sand Samples. Organic Geochemistry. 39: 1210-1214.
Karlsen, D. A., 1991, Larter, .S. R. Analysis of petroleum fractions by TLC-FID: applications to petroleum reservoir description. Organic Geochemistry, 17: 603-617.
Khan, A. Z., and I. K. Brett. 2004, Multi-Dimensional HPLC Determination of Aromatic Core Content, Mass Composition and Aliphatic Side Chain Distributions in Hydrocarbon Fractions of Heavy Distillates. Fuel Chem. Div. Preprints, 49: 487-491
Kharrat, A. M., J. Zacharia, V. J. Cherian, and A. Anyatonwu, 2007. Issues with Comparing SARA Methodologies. Energy and Fuels, 21: 3618-3621.
Lee, S. W., and B. Glavincevski, 1999. NMR Method for Determination of Aromatics in Middle Distillate Oils. Fuel Processing Tech. 60: 81-86.
Lee. S. W., Coulombe, S., and B. Galavincevski. 1990. Investigation of Methods for Determining Aromatics in Middle-Distillate Fuels. Energy Fuels, 4: 20-23.
Lysaght. M. J., Kelly, J. J., and J. B. Callis. 1993. Rapid Spectroscopic Determination of Percent Aromatics. Percent Saturates and Freezing Point of JP-4 Aviation Fuel. Fuel. 72: 623-631.
Masson. J-F., T. Price, and P. Collins. 2001, Dynamics of Bitumen Fractions by Thin-Layer Chromatography/Flame Ionization Detection. Energy and Fuel, 15: 955-960.
Michaelian, K. H., Zhang, S. L., Hall. R. H., and J.T. Bulmer, 2001. Fourier Transform Raman Spectroscopy of Syncrude Sweet Blend Distillation Fractions Derived from Athabasca Bituemn. Spectrochimicta Acta A., 57: 73-81.
Mitchell, D. L., and J. G. Speight. 1973, The Solubility of Asphaltenes in Hydrocarbon Solvents. Fuel, 52, 149-152.
Orea, M., Alberdi. M., Ruggiero, A., and S. Colaiocco, 2002. Alternative Calibration and Standardization Procedure of latroscan TLC-FID for SARA Hydrocarbon Class Quantification: Application to TAR-MAT Zone Identification. Fuel Chem. Div, Preprints, 47: 652-655.
Oyekunle, L. O., 2007. Influence of Chemical Composition on the Physical Characteristics of Paving Asphalts. Pet. Sci. and Tech. 25: 1401-1414.
Radke, M., H. Willsch, and H. Welte, 1980, Preparative Hydrocarbon Group Type Determination by automated Medium Pressure Liquid Chromatography. Analytical Chemistry, 52: 406-411.
Ramaswamy, V., Kothiyal, V., Singh, I. D., and R. Krishna, 1988, Estimation of Total Aromatics in Kerosene Fractions by Infrared Spectroscopy-II. J, Chem, Tech. Biotechnol. 41: 255-259
Robbins, W. K., 1998. Quantitative Measurement of Mass and Aromaticity Distributions for Heavy Distillates 1. Capabilities of the HPLC-2 System. J. Chromatographic. Sci., 36: 457 466.
Rudzinski, W. E., and T.M. Aminabhavi, 2000, A Review on Extraction and Identification of Crude Oil and Related Products using Supercritical Fluid Technology. Energy Fuels, 14: 464-475.
Rudzinski, W. E., Aminabhavi, T. M., Sassman, S., and L. M. Watkins. 2000. Isolation and Characterization of the Saturate and Aromatic Fractions of a Maya Crude Oil. Energy Fuels, 14: 839-844.
Schabron, J. F., and J. F. Rovani, Jr., 2008. On-Column Precipitation and Re-Dissolution of Asphaltenes in Petroleum Residua, Fuel, 87: 165-176.
Schabron, J. F., J. F. Rovani, and M. M. Sanderson, 2010, Asphaltene Determinator Method for Automated On-Column Precipitation and Redissolution of Pericondensed Aromatic Asphaltene Components. Energy and fuels, 24: 5984-5996.
Sjoblom, J., Aske, N., Auflem, I. H., Brandal, O., Havre, T. E., Saether, O., Westvik, A ., Johnsen, E.E., and H. Kallevik. 2003. Our Current Understanding of Water-in-Crude Oil Emulsions. Recent Characterization Techniques and High Pressure Performance. Adv. In Colloid and Interface Sci., 100-102: 399-473.
Suatoni, J. C., Garber, H. R., and B. E. J. Davis. 1975, Hydrocarbon Group Types in Gasoline-Range Materials by High Performance Liquid Chromatography. Chromatographic Sci., 13: 367-371.
Suzuki, T., Itoh, M., Takegami, Y., and Y. Watanabe, 1982, Chemical Structure of Tar-Sand Bitumens by 13C and 1H NMR Spectroscopic Methods. Fuel, 61: 402-410.
Transportation Research Board Special Report 311. 2013, Effects of Diluted Bitumen on Crude Oil Transmission Pipelines.
U.S. Department of Energy Office of Industrial Technologies Report, 1998. “Petroleum Industry of the Future, Energy and Environmental Profile of the U.S. Petroleum Refining Industry” prepared by Energetics. Inc., Columbia, Md.
U.S.E.I.A., 2011, Refinery Capacity 2011, U.S. Energy Information Administration, Washington. D.C. www.eia.gov.
U.S. Pat. No. 4,988,446, Haberman, J. L., Overfield R. E., and W. K. Robbins, 1991. Method for Spectroscopic Analysis of Hydrocarbons.
U.S. Pat. No. 5.348.645 Maggard, S. M., and W. T. Welch. 1994a. Determination of Aromatics in Hydrocarbons by Near Infrared Spectroscopy.
U.S. Pat. No. 5,370,790 Maggard, S. M., and W. T. Welch. 1994b. Determination of Aromatics in hydrocarbons hy Near Infrared Spectroscopy.
U.S. Pat. No. 7.875.464. Schabron. J. F. and J. F. Rovani, 2011. Processing and Analysis Techniques Involving In-Vessel Material Generation.
van de Ven, A. M. C., Jansen. L. and J. den Heijer, 1995. An Infrared Analysis Method to Determine the Aromatic Carbon Content of Mineral Lubricant Based Oils Based on Correlation with Carbon-13 NMR Aromaticities. Lubrication Sci. 7: 197-209.
Vazquez, D., and G. A. Mansoori. 2000, Identification and Measurement of Petroleum Precipitates, J. of Petroleum Sci. and Eng., 26: 49-55.
Westbrook, S. R., and S. A. Hutzler, 1996. The Use of AOTF-NOR Spectrometers to Analyze Fuels Phase I. Instrument Selection and Preliminary Calibrations, Defense Technical Information Center, Interim Report TFLRF No. 313
Wu, W., Saidian. M, Gaur. S., and M. Prasad. 2012. Errors and Repeatability in VSARA Analysis of Heavy Oils. SPE 146107: 1-15.
Storm, D. A., Decanio, S.J. Edwards, J. C., and Sheu, F. Y., 1997. Sediment Formation During Heavy Oil Upgrading, Petroleum Science and Technology. 15(1 &2). 77-102.

Thus, the applicant(s) should be understood to have support to claim and make a statement of invention to at least: i) each of the separation and/or analysis devices/apparatus as herein disclosed and described, ii) the related methods disclosed and described, iii) similar, equivalent, and even implicit variations of each of these devices and methods, iv) those alternative designs which accomplish each of the functions shown as are disclosed and described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such systems or components, ix) each system, method, and element shown or described as now applied to any specific field or devices mentioned, x) methods and apparatuses substantially as described hereinbefore and with reference to any of the accompanying examples, xi) an apparatus for performing the methods described herein comprising means for performing the steps, xii) the various combinations and permutations of each of the elements disclosed, xiii) each potentially dependent claim or concept as a dependency on each and every one of the independent claims or concepts presented, and xiv) all inventions described herein.

In addition and as to computer aspects and each aspect amenable to programming or other electronic automation, the applicant(s) should be understood to have support to claim and make a statement of invention to at least: xv) processes performed with the aid of or on a computer as described throughout the above discussion, xvi) a programmable apparatus as described throughout the above discussion, xvii) a computer readable memory encoded with data to direct a computer comprising means or elements which function as described throughout the above discussion, xviii) a computer configured as herein disclosed and described, xix) individual or combined subroutines and programs as herein disclosed and described, xx) a carrier medium carrying computer readable code for control of a computer to carry out separately each and every individual and combined method described herein or in any claim, xxi) a computer program to perform separately each and every individual and combined method disclosed, xxii) a computer program containing all and each combination of means for performing each and every individual and combined step disclosed, xxiii) a storage medium storing each computer program disclosed, xxiv) a signal carrying a computer program disclosed, xxv) the related methods disclosed and described, xxvi) similar, equivalent, and even implicit variations of each of these systems and methods, xxvii) those alternative designs which accomplish each of the functions shown as are disclosed and described, xxviii) those alternative designs and methods which accomplish each of the functions shown as are implicit to accomplish that which is disclosed and described, xxix) each feature, component, and step shown as separate and independent inventions, and xxx) the various combinations and permutations of each of the above.

With regard to claims whether now or later presented for examination, it should be understood that for practical reasons and so as to avoid great expansion of the examination burden, the applicant may at any time present only initial claims or perhaps only initial claims with only initial dependencies. The office and any third persons interested in potential scope of this or subsequent applications should understand that broader claims may be presented at a later date in this case, in a case claiming the benefit of this case, or in any continuation in spite of any preliminary amendments, other amendments, claim language, or arguments presented, thus throughout the pendency of any case there is no intention to disclaim or surrender any potential subject matter. It should be understood that if or when broader claims are presented, such may require that any relevant prior art that may have been considered at any prior time may need to be re-visited since it is possible that to the extent any amendments, claim language, or arguments presented in this or any subsequent application are considered as made to avoid such prior art, such reasons may be eliminated by later presented claims or the like. Both the examiner and any person otherwise interested in existing or later potential coverage, or considering if there has at any time been any possibility of an indication of disclaimer or surrender of potential coverage, should be aware that no such surrender or disclaimer is ever intended or ever exists in this or any subsequent application. Limitations such as arose in Hakim v. Cannon Avent Group, PLC, 479 F.3d 1313 (Fed. Cir 2007), or the like are expressly not intended in this or any subsequent related matter. In addition, support should be understood to exist to the degree required under new matter laws—including but not limited to European Patent Convention Article 123(2) and United States Patent Law 35 USC 132 or other such laws—to permit the addition of any of the various dependencies or other elements presented under one independent claim or concept as dependencies or elements under any other independent claim or concept. In drafting any claims at any time whether in this application or in any subsequent application, it should also be understood that the applicant has intended to capture as full and broad a scope of coverage as legally available. To the extent that insubstantial substitutes are made, to the extent that the applicant did not in fact draft any claim so as to literally encompass any particular embodiment, and to the extent otherwise applicable, the applicant should not be understood to have in any way intended to or actually relinquished such coverage as the applicant simply may not have been able to anticipate all eventualities; one skilled in the art, should not be reasonably expected to have drafted a claim that would have literally encompassed such alternative embodiments.

Further, if or when used, the use of the transitional phrase “comprising” is used to maintain the “open-end” claims herein, according to traditional claim interpretation. Thus, unless the context requires otherwise, it should be understood that the term “comprise” or variations such as “comprises” or “comprising”, are intended to imply the inclusion of a stated element or step or group of elements or steps but not the exclusion of any other element or step or group of elements or steps. Such terms should be interpreted in their most expansive form so as to afford the applicant the broadest coverage legally permissible. The use of the phrase, “or any other claim” is used to provide support for any claim to be dependent on any other claim, such as another dependent claim, another independent claim, a previously listed claim, a subsequently listed claim, and the like. As one clarifying example, if a claim were dependent “on claim 20 or any other claim” or the like, it could be re-drafted as dependent on claim 1, claim 15, or even claim 25 (if such were to exist) if desired and still fall with the disclosure. It should be understood that this phrase also provides support for any combination of elements in the claims and even incorporates any desired proper antecedent basis for certain claim combinations such as with combinations of method, apparatus, process, and the like claims.

Finally, any claims set forth at any time are hereby incorporated by reference as part of this description of the invention, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice-versa as necessary to define the matter for which protection is sought by this application or by any subsequent continuation, division, or continuation-in-part application thereof, or to obtain any benefit of, reduction in fees pursuant to, or to comply with the patent laws, rules, or regulations of any country or treaty, and such content incorporated by reference shall survive during the entire pendency of this application including any subsequent continuation, division, or continuation-in-part application thereof or any reissue or extension thereon.

What is claimed is:

1. A method for analyzing a pre-treatment hydrocarbon that comprises volatiles, said method comprising the steps of:
treating said pre-treatment hydrocarbon by segregating at least a portion of said volatiles from said pre-treatment hydrocarbon, under a vacuum, without oxidizing said pre-treatment hydrocarbon, without cracking said pre-treatment hydrocarbon, and while said pre-treatment hydrocarbon is heated to a temperature of more than or equal to 420 degs. C. atmospheric equivalent boiling point;
generating a hydrocarbon residue and segregated hydrocarbon volatiles; and
analyzing at least one of said hydrocarbon residue and said segregated hydrocarbon volatiles.

2. A method as described in claim 1 wherein said step of analyzing at least one of said hydrocarbon residue and said segregated hydrocarbon volatiles comprises the step of analyzing said hydrocarbon residue.

3. A method as described in claim 2 wherein said step of analyzing said hydrocarbon residue comprises the step of analyzing with an evaporative analysis that would evaporate said portion of said volatiles if said volatiles were subjected to said evaporative analysis.

4. A method as described in claim 3 wherein said step of segregating at least a portion of said volatiles comprises the step of segregating at least substantially all components of said hydrocarbon that would evaporate if subjected to said evaporative analysis.

5. A method as described in claim 2 wherein said step of segregating at least a portion of said volatiles comprises the step of segregating enough of said volatiles so that results of said step of analyzing said hydrocarbon residue are not unacceptably negatively impacted.

6. A method as described in claim 1 wherein said step of segregating at least a portion of said volatiles comprises the step of segregating at least a heavier portion of said volatiles under said vacuum.

7. A method as described in claim 6 wherein said step of segregating said at least said heavier portion of said volatiles under a vacuum comprises the step of vacuum distilling.

8. A method as described in claim 7 wherein said step of vacuum distilling comprises the step of vacuum distilling while said pre-treatment hydrocarbon is heated to a temperature of less than or equal to 340 degs. C.

9. A method as described in claim 7 wherein said step of vacuum distilling comprises the step of vacuum distilling while said pre-treatment hydrocarbon is under a vacuum of from 0.00005-30 mm Hg.

10. (canceled)

11. A method as described in claim 1 wherein said step of segregating said volatiles under a vacuum comprises the step of vacuum transferring a lighter portion of said volatiles and vacuum distilling a heavier portion of said volatiles.

12. A method as described in claim 1 wherein said step of segregating said volatiles from said pre-treatment hydrocarbon comprises the step of cooling said volatiles.

13. A method as described in claim 12 wherein said step of cooling said volatiles comprises the step of cooling said volatiles using a technique selected from the group consisting of: cooling bath, dry ice cooling bath, dry ice acetone cooling bath, salt and ice and acetone cooling bath, slurry made with solvents and dry ice cooling bath, slurry made with solvents and liquid nitrogen cooling bath, liquid nitrogen cooling bath, liquid helium cooling bath, and liquid helium solvent slurry cooling bath.

14. A method as described in claim 12 wherein said step of cooling comprises the step of cooling with an electric chiller.

15. A method as described in claim 12 wherein said step of cooling comprises cooling a collection vessel that contains said segregated volatiles.

16. A method as described in claim 1 wherein said pre-treatment hydrocarbon comprises a hydrocarbon selected from the group consisting of: oil, crude oil, biofuel, petroleum oil, shale oil, coal-derived oil, synthetic oil, vegetable oil, nut oil, fossil oil, biomass derived oil, oil with additive, oil constituent, asphalt binder, dilbit, opportunity crude oil, extra heavy oil, high TAN crude, solvent diluted oil, undiluted oil, oil from environmental release, pollutant oil, lubrication oil, recycled oil, asphalt material, tar sands bitumen, feed oil, asphalt, coal liquid, bitumen, crude oil, light crude oil, medium crude oil, heavy crude oil, medium crude oil, extra heavy crude oil, cracked hydrocarbon, partially cracked hydrocarbon, synthetic crude oil, blended crude oil, food grade oil, and oil-base cosmetics.

17. A method as described in claim 1 wherein said pre-treatment hydrocarbon is a very low asphaltene content oil that is not amenable to conventional Heithaus or other flocculation titration methods.

18. (canceled)

19. A method as described in claim 2 wherein said step of analyzing said hydrocarbon residue comprises the step of analyzing using a technique selected from the group of techniques consisting of: evaporative light scattering detector (ELSD), charged aerosol detector (CAD), evaporative method, on-column precipitation method, re-dissolution method, Asphaltene Determinator (AD), Waxphaltene Determinator (WD), Saturates Aromatics Resins-Asphaltene Determinator (SAR-AD), adsorbent column separations of saturates, aromatics, resins, and asphaltenes, nuclear magnetic resonance (NMR) spectroscopy, one-dimensional gas chromatography, two-dimensional gas chromatography, multi-dimensional gas chromatography, gas chromatography/mass spectrometry (GC/MS), high performance liquid chromatography (HPLC), ion exchange chromatography, fluorescence spectroscopy, turbidimetric spectroscopy, ultraviolet (UV) spectroscopy, ultraviolet visible (UV-Vis) spectroscopy, infrared spectroscopy (IR), Fourier transform infrared spectroscopy (FTIR), attenuated total reflectance infrared spectroscopy (ATR-IR), Raman spectroscopy, near infrared spectroscopy, acousto-optic tunable filter near infrared spectroscopy (AOTF-NIR), Fourier transfer Raman spectroscopy, high resolution Fourier transform ion cyclotron resonance mass spectrometry (FTICR/MS),optical microscopy, X-ray microscopy, X-ray fluorescence spectroscopy, refractive index, supercritical fluid chromatography, supercritical fluid extraction, electrical resistivity, differential scanning calorimetry (DSC), thermogravimetric analysis (TGA), titration, acid-base titration, and flocculation titration.

20. A method as described in claim 1 wherein said step of analyzing at least one of said hydrocarbon residue and said segregated hydrocarbon volatiles comprises the step of analyzing said segregated hydrocarbon volatiles.

21. A method as described in claim 20 wherein said step of analyzing said segregated hydrocarbon volatiles comprises the step of analyzing using a technique selected from the group of techniques consisting of: NMR spectroscopy, gas chromatography, one-dimensional gas chromatography, two-dimensional gas chromatography, multi-dimensional gas chromatography,GC/MS, HPLC, UV spectroscopy, UV-Vis spectroscopy, IR spectroscopy, spectroscopy, ATR-IR, FT-IR spectroscopy, NIR spectroscopy, AOTF-NIR spectroscopy, FTICR/MS, optical fluorescence spectroscopy, turbidimetric spectroscopy, x-ray fluorescence spectroscopy, refractive index, supercritical fluid chromatography, supercritical extraction, titration, acid-base titration, and electrical resistivity.

22. A method as described in claim 21 wherein said step of analyzing said segregated hydrocarbon volatiles comprises the step of analyzing hydrocarbon volatiles distillate.

23. A method as described in claim 1 wherein said step of analyzing at least one of said hydrocarbon residue and said segregated hydrocarbon volatiles comprises the step of analyzing said hydrocarbon residue and said segregated hydrocarbon volatiles.

24. A method as described in claim 23 further comprising the step of comparing results generated by said step of analyzing said hydrocarbon residue with results generated by said step of analyzing said segregated hydrocarbon volatiles.

25. A method as described in claim 1 wherein said step of analyzing at least one of said hydrocarbon residue and said segregated hydrocarbon volatiles comprises the step of analyzing said hydrocarbon residue and analyzing said segregated hydrocarbon volatiles.

26. A method as described in claim 25 wherein said step of analyzing said hydrocarbon residue comprises the step of generating hydrocarbon residue analysis results and said step of analyzing said segregated hydrocarbon volatiles comprises the step of generating volatiles analysis results.

27. A method as described in claim 26 further comprising the step of using said hydrocarbon residue analysis results and said hydrocarbon volatiles analysis results to generate improved analysis results for said pre-treatment hydrocarbon.

28. A method as described in claim 27 wherein said improved analysis results are selected from the group consisting of colloidal stability index results, asphaltene stability index results, Gaestel index results, and resins to asphaltene ratio results.

29. A method as described in claim 27 further comprising the step of using said improved analysis results to improve predictability or control of one or more phenomena, wherein said phenomena are selected from the group consisting of emulsion-related effects, emulsion formation, emulsion formulation, breaking of emulsions, fouling generally, heat exchanger fouling, distillation tower fouling, catalyst fouling, catalyst efficiency, oil subfraction measurement, settling, asphaltene deposition, asphaltene precipitation, distillation efficiency, coke formation, corrosion, sediment formation and asphaltene adsorption.

30. A method as described in claim 27 further comprising the step of using said improved analysis results to improve control of at least one process.

31-32. (canceled)

33. A method as described in claim 1 further comprising the step of determining whether olefins are present in said pre-treatment hydrocarbon.

34. A method as described in claim 1 further comprising determining the stability of a pyrolytic process to which said pre-treatment hydrocarbon has been subjected.

35. A method as described in claim 34 wherein said pyrolytic processes comprises a process selected from the group consisting of: thermal cracking, catalytic cracking, hydrocracking, coking, and thermal upgrading.

36. A method as described in claim 1 further comprising the step of characterizing said pre-treatment hydrocarbon.

37. A method as described in claim 36 wherein said step of characterizing said pre-treatment hydrocarbon comprises the step of identifying said pre-treatment hydrocarbon as being a hydrocarbon selected from the group consisting of: bitumen, heavy oil blended with a light diluent, medium oil, extra heavy oil, a blend thereof, heavy oil or bitumen blended with a light oil, and heavy oil or bitumen blended with other processed streams.

38. A method as described in claim 1 further comprising the step of monitoring a thermal cracking and upgrading process to which said pre-treatment hydrocarbon has been subjected.

39. A method as described in claim 1 further comprising the step of determining relative concentrations of acids in the segregated hydrocarbon volatiles and hydrocarbon residue.

40. A method of analyzing a hydrocarbon that comprises volatiles having a first volatiles portion and a second volatiles portion, wherein volatile components of said first volatiles portion have boiling points that are less than or equal to a first portion boiling point maximum; and wherein volatile components of said second volatiles portion have boiling points that are less than or equal to a second portion boiling point maximum that is higher than said first portion boiling point maximum, said method comprising the steps of:
segregating at least some of said first volatiles portion from said hydrocarbon;
segregating at least some of said second volatiles portion of said hydrocarbon while said hydrocarbon is under a vacuum;
wherein both said steps of segregating are performed without oxidizing or cracking said hydrocarbon,
generating hydrocarbon residue and segregated hydrocarbon volatiles; and
analyzing at least one of said hydrocarbon residue and said segregated hydrocarbon volatiles.

41-82. (canceled)

83. A method of improving the results of conventional hydrocarbon analysis, said conventional hydrocarbon analysis effecting evaporation of at least some of any volatiles present in a hydrocarbon during said analysis thereof, said method comprising the steps of:
segregating at least some volatiles from a sample of a pre-treatment hydrocarbon while said sample is under a vacuum and an inert atmosphere, and while said pre-treatment hydrocarbon is heated to a temperature of more than or equal to 420 degs. C. atmospheric equivalent boiling point, thereby generating a hydrocarbon residue and segregated hydrocarbon volatiles;
analyzing said hydrocarbon residue via said conventional hydrocarbon analysis to generate hydrocarbon residue analysis results,
analyzing said segregated hydrocarbon volatiles to generate hydrocarbon volatiles analysis results,
using said hydrocarbon residue analysis results and said hydrocarbon volatiles analysis results to generate improved analysis results for said pre-treatment hydrocarbon as compared to those results that conventional hydrocarbon analysis on said pre-treatment hydrocarbon.

84-234. (canceled)
